SEQUENCE LISTING (4 sequences)

SEQ ID NO: 1 - DNA, 20 bp, Vaccinia virus
  misc_feature (1)..(20): corresponds to nucleotides 268-284 of GenBank AJ269556
ttcggaggcacaatgaatga

SEQ ID NO: 2 - DNA, 25 bp, Vaccinia virus
  misc_feature (1)..(25): reverse primer corresponds to nucleotides 312-288 of GenBank AJ269556
tgtcttcaataggaggacactttgc

SEQ ID NO: 3 - DNA, 17 bp, Vaccinia virus
  misc_feature (1)..(17): corresponds to nucleotides 268-284 of GenBank AJ269556 PUBLICATION INFORMATION
ctctaggagagccattc

SEQ ID NO: 4 - DNA, 17 bp, Artificial
  internal control
ctctaggacattcagcg

(19) United States
(12) Patent Application Publication
(10) Pub. No.: US 2006/0110407 A1
(43) Pub. Date: May 25, 2006

(54) Immune globulin formulations for the treatment and prevention of an orthopoxvirus infection

(76) Inventors: Shelley Stopera, Manitoba (CA); Maurice Genereux, Manitoba (CA); Mauricio Edge, Manitoba (CA)

Correspondence Address: Ade & Company, 1700-360 Main Street, Winnipeg Manitoba, R3C 3Z3, CA

(21) Appl. No.: 10/476,853

(22) PCT Filed: Dec. 5, 2002
(86) PCT No.: PCT/CA02/01866

§ 371 Date: Apr. 2, 2004

Related U.S. Application Data
(60) Provisional application No. 60/335,828, filed on Dec. 5, 2001.
(60) Provisional application No. 60/350,878, filed on Jan. 25, 2002.

(51) Int. Cl. C12Q 1/70; C12P 21/04; A61K 39/275; C07K 16/10; A61K 39/285

(52) U.S. Cl. 424/232.1; 435/70.21; 435/5; 530/388.3

(57) ABSTRACT

The invention provides an immune globulin having a high titre of antibody to Orthopoxvirus, pharmaceutical compositions comprising the immune globulin and methods for making same. In one embodiment the immune globulin is intravenously injectable. The invention also provides a colorimetric assay to detect neutralizing antibodies to vaccinia virus. The invention has a number of uses including detection of neutralizing antibodies to vaccinia virus and the immunization of persons against the Orthopoxvirus and in the treatment of related conditions.

This application claims priority under 35 USC § 119(e) to Provisional Patent Application Ser. No. 60/335,828, filed Dec. 5, 2001 and to Provisional Patent Application Ser. No. 60/350,878, filed Jan. 25, 2002.

This invention relates to an immune globulin and methods for its production and use. In one aspect, the invention relates to an immune globulin preparation suitable for use in humans and having an antibody to viruses belonging to the genus Orthopoxvirus.

### BACKGROUND OF THE INVENTION

### Vaccination

Currently, international concern is heightened regarding the use of Smallpox virus as a bioterrorism agent. Smallpox is a disease caused by an infection with the variola virus, a member of the genus Orthopoxvirus. The last naturally occurring case of Smallpox was reported in Somalia in 1977. Since recommendations for routine Smallpox vaccination were rescinded in North America and most of Europe in 1971, and the effectiveness of vaccination appears to last only 10 years, much of the world population is currently susceptible to infection. There are exceptions such as the former Soviet Union where vaccine production has continued as well as vaccination of the general public. During the Smallpox era, overall mortality rates were approximately 30%. Death usually occurred late in the first week or during the second week of illness and was usually attributed to overwhelming viremia. The virus is highly transmissible from person-to-person and infected individuals may, in turn, infect tens to hundreds of susceptible contacts.

To date, there is no proven treatment for human variola virus infections. The only known method of preventing infection with variola virus is by vaccination with a less deadly strain of Orthopoxvirus such as vaccinia virus. This is possible because there is antigenic cross reactivity between viral surface antigens within the Orthopoxvirus genus. Vaccinia virus is the prototype of the genus Orthopoxvirus. It is a double-stranded DNA (deoxyribonucleic acid) virus that has a broad host range under experimental conditions but is rarely isolated from animals outside the laboratory (Fenner F, et al., Orthopox Viruses. San Diego, Calif.: Academic Press, Inc., 1989; Damaso C R A, et al. Virology 2000;277:439-49.). Multiple strains of vaccinia virus exist that have different levels of virulence for humans and animals. For example, the Temple of Heaven and Copenhagen vaccinia strains are highly pathogenic among animals, whereas the NYCBOH (New York City Board of Health) strain, from which the Wyeth vaccine strain was derived, exhibits relatively low pathogenicity (Fenner F, et al, Geneva, Switzerland: World Health Organization, 1988.).

The vaccinia vaccine is administered intradermally using a bifurcated needle. After primary vaccination, neutralizing antibodies are detectable at about the tenth day. Successful primary vaccination results in virus proliferation in the epidermis leading to papule development in three to five days. The papule then evolves into a pustule that attains its maximum size after 8 to 12 days. The scab forms and separates at 14 to 21 days leaving a typical vaccination scar. Low-grade fever usually accompanies pustule development as well as tenderness due to swelling of the draining local lymph nodes. Viremias may occur within the first week, and in certain individuals, viremias may persist for longer periods of time.

### Complications of Vaccination

After vaccination, three types of abnormal skin reactions may occur: eczema vaccinatum, progressive vaccinia and generalized vaccinia.

Eczema vaccinatum is a localized or systemic dissemination of vaccinia virus among persons who have eczema or other chronic exfoliative skin conditions. The illness is usually mild but may be more severe and is occasionally fatal (Henderson and Moss, Smallpox and Vaccinia. In Vaccines (S. A. Plotkin and W. A. Orenstein, eds), 1999 p. 74-97).

Progressive vaccinia is a severe, potentially fatal illness that occurs mainly among persons with cellular immunodeficiencies. It is characterized by a progressive necrosis in the area of vaccination (Henderson and Moss, Smallpox and Vaccinia. In Vaccines (S. A. Plotkin and W. A. Orenstein, eds), 1999 p. 74-97).

Generalized vaccinia is characterized by one or more lesions that develop after vaccination at locations other than the vaccination site. The rash is typically self-limiting and requires no therapy except among patients with serious underlying illnesses (Henderson and Moss, Smallpox and Vaccinia. In Vaccines (S. A. Plotkin and W. A. Orenstein, eds), 1999 p. 74-97).

Accidental inoculation of vaccinia virus transferred from the vaccination site lesion is quite common, accounting for about 50% of all complications of primary vaccination and revaccination. The most common sites for inoculation are the face, eyelid, nose, mouth, genitalia and rectum. Such lesions usually heal at the same time as the primary lesion (Morbid. Mortal. Weekly Rep 1991;40(RR14):1-10).

The most serious complication of vaccinia vaccination is postvaccinial encephalitis. Approximately 15 to 25% of individuals with this complication die. Recovery is seldom complete and the patient is left with mental impairment and some degree of paralysis (Lane et al., 1970, J. Infect. Dis. 122:303-309).

### Treatment of Complications from Vaccination

Immune globulins, typically fractionated from donor plasma and having high titers of a particular antibody, are therapeutically useful in treating patients deficient or in need of a such antibody. Such immune globulin are typically formulated into a pharmaceutical formulation. The only product that is available for treating complications associated with Smallpox vaccination is vaccinia immune globulin (VIG-IM) which may only be administered via intramuscular injection. VIG-IM is a sterile solution of the immune globulin fraction(s) isolated from the plasma of persons previously vaccinated with vaccinia vaccine (Morbid. Mortal. Weekly Rep 1991;40(RR14):1-10).

VIG-IM has shown some effectiveness in treating eczema vaccinatum, some cases of progressive vaccinia and is also useful for treating ocular vaccinia resulting from inadvertent inoculation. In cases where the patient has a toxic condition or serious underlying disease, VIG-IM is also recommended to treat severe generalized vaccinia. VIG-IM treatment is not beneficial for postvaccinial encephalitis.

There is no current supply or manufacturer of licensed VIG-IM in North America. In addition to its expired status, the only available VIG-IM has many drawbacks. First, the VIG-IM is manufactured using the Cohn ethanol fractionation method, which results in the presence of several antibody classes and subtypes, including IgG₄ and IgA. The presence of IgA in immune globulin preparations administered to patients has resulted in anaphylactic shock in certain individuals. The Cohn method of preparing immune globulins also results in aggregates and fragmentation of antibodies, which will fix complement. The presence of these aggregates and fragments precludes the VIG-IM from being administered intravenously (IV), and necessitates administration via intramuscular (IM) injection.

The recommended dosage of the currently available VIG-IM for treatment of complications resulting from vaccinia virus vaccination is 0.6 mL/kg of body weight. A person weighing 70 kg will require a 42 mL intramuscular injection. Intramuscular injection of such large volumes of fluid is very painful, and may be difficult in certain individuals with little muscle mass, such as infants, children and the elderly. Due to this large volume, the does is typically administered in multiple injections at different injection sites, thereby complicating the therapeutic regimen.

### Orthopoxviruses That Infect Humans

The genus Orthopoxvirus includes Buffalopox, California vole pox, camelpox, cowpox, ectromelia, monkeypox, rabbitpox, raccoon pox, tatera pox, Uasin Gishu pox, Vaccinia, variola, and vole pox virus. At least four known species infect humans, namely variola, vaccinia, cowpox and monkeypox. Cross-protection has been demonstrated in experimental animals as well as extensive cross-reactivity in serological tests [Kitamoto N, et al, Arch Virol 1986 91:357-66, Fenner, F. Poxviruses. In Fields Virology (B. N. Fields, D. M. Knipe, P. M. Howley, eds.). 1996, 2673-2699.]. Thus, an immune globulin raised against one member of the Orthopoxvirus family may be beneficial in treating patients infected with other members of the Orthopoxvirus family.

Until its global eradication in 1977, Smallpox was considered to be the most important poxvirus causing infections in man. Recent increases in monkeypox infections in humans may reflect reduced herd immunity resulting from the cessation of widespread Smallpox vaccinations in the early 1970s.

With the potential threat of bioterrorism using variola virus or related natural or recombinant Orthopoxviruses, and the natural emergence of other members of the Orthopoxvirus members as potential threats to humans, a clear need exists for a therapy or treatment for individuals infected with an Orthopoxvirus. Should the need arise to vaccinate or re-vaccinate individuals with vaccinia vaccine, the quantifiable possibility of adverse reactions remains. There clearly exists a need for an Orthopoxvirus immune globulin product with less complications than VIG-IM and which can be administered in a route other than intramuscularly.

An intravenously injectable Orthopoxvirus immune globulin product produced from human plasma may not be feasible as routine vaccination against Smallpox ceased in most of the world in 1970. Additionally, donor plasma is screened of a variety of pathogens that were not screened for in the 1980's, such as HIV-1 and HIV-2, Hepatitis C, and human T-cell lymphoma virus 1 and 2. As such, modern plasma differs in composition from the plasma fractionated in the 1980's.

An immune globulin formulation for intravenous injection differs from an immune globulin formulation for intramuscular injection in that the former has low levels of aggregated immune globulin and low levels of complement fixing activity. The exact mode of action of a immune globulin after it is injected into a patient is not clearly understood. As such, it is not known if an Orthopoxvirus immune globulin, formulated for intravenous injection with low aggregate concentrations, low complement fixing activity and from donor plasma that differs in composition from plasma in the 1980's, will be efficacious against Orthopoxvirus infections as an intramuscular formulation. Intravenous immune globulin formulations have not been effective against against many viral infections including Hepatitis B (that Wahl M, et al J Hepatol (1989) 9:198-203), Hepatitis C (Liddle C, Anaesth Intensive Care (1996) 24:180-3), HIV (Prince A M, H, et al, Proc Natl Acad Sci USA (1988) 85:6944-8), cytomegalovirus infection (Toivanen A, and Wahren B Bone Marrow Transplant (1987) 2:299-305), and SIV (Almond N, et al, J Gen Virol (1997) 78 (Pt 8):1919-22).

### SUMMARY OF THE INVENTION

It is one object of the present invention to obviate or mitigate some of the above noted limitations.

According to a first aspect of the invention, there is provided a method of preparing an intravenously injectable immune globulin effective against Orthopoxvirus comprising:

vaccinating a plurality of donors with an Orthopoxvirus vaccine;

isolating plasma from each of said donors after a period of time sufficient to allow production of antibodies against said Orthopoxvirus vaccine; and

preparing an intravenously injectable immune globulin from the plasma.

According to a second aspect of the invention, there is provided a method of treating or preventing an Orthopoxvirus infection in an individual comprising administering intravenously to an individual in need of such treatment an intravenously injectable immune globulin effective against Orthopoxvirus.

According to a third aspect of the invention, there is provided a method of treating or ameliorating symptoms associated with adverse reaction to Orthopoxvirus vaccination comprising administering intravenously to an individual in need of such treatment an intravenously injectable immune globulin effective against Orthopoxvirus.

According to a fourth aspect of the invention, there is provided an intravenously injectable pharmaceutical composition comprising immune globulin effective against Orthopoxvirus.

According to a fifth aspect of the invention, there is provided a colorimetric assay for measuring vaccinia virus neutralizing antibodies comprising:

adding a quantity of vaccinia virus to a sample suspected of containing vaccinia neutralizing antibodies, thereby forming a mixture;

incubating the mixture;

adding the mixture to cultured animal cells susceptible to vaccinia virus infection; and

adding a vital dye to the cells,

wherein the amount of neutralizing antibodies present in the sample is proportional to staining of the cultured cells by the vital dye.

According to a sixth aspect of the invention, there is provided a pharmaceutical kit for treatment of a Orthopoxvirus infection in a subject in need thereof, the kit comprising in packaged combination: an immune globulin effective against an Orthopoxvirus, and an antiviral agent effective against an Orthopoxvirus.

According to a seventh aspect of the invention, there is provided a method of testing a sample for vaccinia DNA comprising:

providing a sample suspected of containing vaccinia DNA under conditions suitable for oligonucleotide;

adding to said sample reagents needed for nucleotide amplification, said reagents including an enzyme capable of synthesizing nucleotide molecules, buffers and appropriate substrates for nucleotide synthesis, said reagents further comprising:
a forward primer comprising an oligonucleotide molecule binding to a first region of Vaccinia virus DNA;
a reverse primer comprising an oligonucleotide molecule binding to a second region of Vaccinia virus DNA, said second region being sufficiently proximal to the first region that an amplification product may be produced; and
a reporter primer comprising a reporter oligonucleotide molecule binding to a third region of Vaccinia virus DNA, said third region being between the first region and the second region, said reporter primer further comprising a reporter molecule attached to a first end of the reporter oligonucleotide and an effector molecule attached to a second end of the reporter oligonucleotide, said effector molecule altering a signal generated by the reporter molecule when the effector molecule and the reporter molecule are both attached to the reporter oligonucleotide; and
   incubating the sample and reagents under conditions suitable for nucleotide amplification by the enzyme.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS AND TABLES

FIG. 1 shows Bivariate Fit of log PRNT By Log ELISA.

FIG. 2 shows average titers over time.

FIG. 3 shows the mean restimulation titers.

Table 1: Adverse effects recorded in patients following vaccination with Dryvax®. 20 patients with previous record of vaccination, as recorded in visits to physicians.

Table 2: Efficacy of vaccinia immune globulin and antiviral agent in treating experimental vaccinia infection. Mice injected with vaccinia virus and subsequently treated with either VIG, Cidofovir™ or both. Pocks on mouse tail were counted one week following treatment.

### DETAILED DESCRIPTION OF THE INVENTION

Described herein is a method of obtaining immune globulins from an individual inoculated with an Orthopoxvirus vaccine, the purified immune globulin obtained therefrom an a number of medical uses thereof.

A colorimetric assay has been developed to detect neutralizing antibodies to vaccinia virus. Basically, Orthopoxvirus antibody preparation, serum, plasma or other biological solutions to be assayed for vaccinia virus neutralizing antibodies are serially diluted. The dilutions are incubated with a given amount of vaccinia virus for a given amount of time. Presumably, this incubation allows neutralizing antibodies, if present, to bind to the vaccinia virus and prevent the virus from infecting cells in subsequent steps of the method. The vaccinia virus/neutralizing antibody mixture is then added to cultured animal cells that are susceptible to infection with vaccinia virus. After incubating the vaccinia virus and cultured cells under conditions that allow the virus to infect the cells, a vital dye is added to the cultured cells. The amount of neutralizing antibody present is proportional to the levels of living cells remaining in the culture, as determined by a vital dye stain.

An Orthopoxvirus antibody preparation has been developed for intravenous injection into patients. One method of producing the Orthopoxvirus antibody preparation is by immunizing animals with an Orthopoxvirus vaccine, followed by collecting plasma or serum from the animals. The plasma or serum may be tested for the levels of class-specific Orthopoxvirus antibodies prior to fractionation of the plasma to obtain immune globulins, such as immune globulin G. The immune globulins are then formulated into pharmaceutical compositions for administration into animals such as humans. Such pharmaceutical compositions are not limited to containing antibodies fractionated from plasma, and may include polyclonal or monoclonal Orthopoxvirus antibodies, or fragments of such antibodies. Such antibodies or fragments of antibodies may also be collected from cells producing such proteins in tissue culture.

A protocol is described for the treatment of patients with an Orthopoxvirus infection or suffering from complications of side-effects of an Orthopoxvirus vaccination. Such an infection may be the result of a variety of causes, as described below. The treatment protocol is the administration of VIG at doses effective against an Orthopoxvirus infection. As will be appreciated by one of skill in the art, it is imperative that the VIG is safe for administration, that is, can be administered with minimal risk of complications or side-effects. As discussed herein, the plasma collected must be substantially free of blood-borne pathogens so that the VIG can be safely administered to individuals in need thereof. As will be appreciated by one of skill in the art, in this context, “substantially free of blood-borne pathogens” indicates that, for example known or emerging pathogens are not present in the donor plasma and VIG at concentrations at what is considered unsafe or unacceptable within the art.

As discussed herein, the VIG may be administered in combination with other compounds. For example, the VIG may be administered simultaneously with or subsequent to vaccination with an Orthopoxvirus vaccine for ameliorating or treating complications associated with Orthopoxvirus vaccination, as discussed herein. In other embodiments, the VIG may be combined with pharmaceutical compositions known in the art for treating or preventing Infections by DNA viruses, for example, Cidofovir™.

### DEFINITIONS

Orthopoxvirus—a virus belonging to the genus Orthopoxvirus. Known viruses include Buffalopox, California vole pox, camelpox, cowpox, ectromelia, monkeypox, rabbitpox, raccoon pox, tatera pox, Uasin Gishu pox, vaccinia, variola, and vole pox virus.
Orthopoxvirus antigen—an antigen that elicits the production of Orthopoxvirus antibodies if administered in a suitable manner to an suitable animal species. Said suitable manner and suitable species would be known to those skilled in the art. In one embodiment, said Orthopoxvirus antigen is preferably expressed on the envelope or surface of an Orthopoxvirus.
Vaccinia virus antibodies—antibodies that can bind and/or neutralize vaccinia virus, said antibodies may also bind and/or neutralize other viruses belonging to the genus Orthopoxvirus, such as cowpox, monkeypox and variola virus. Such vaccinia virus antibodies may be polyclonal and produced in a animal in an immune response to a vaccinia virus vaccine or infection with a vaccinia virus.
Effective against Orthopoxvirus—means prevents or attenuates an Orthopoxvirus infection in an animal.
Effective amount—an amount effective, at dosages and for periods of time necessary to achieve the desired result.
Orthopoxvirus antibodies—antibodies that can bind to an Orthopoxvirus antigen and/or neutralize a virus belonging to the genus Orthopoxvirus. Such Orthopoxvirus antibodies may be polyclonal, and produced in a animal in an immune response to an Orthopoxvirus vaccine or infection with an Orthopoxvirus. Orthopoxvirus antibodies and their fragments may also be produced by a animal cell in cell culture, or by a microorganism using recombinant DNA technology.
Orthopoxvirus vaccine—a vaccine containing an Orthopoxvirus antigen used to immunize an animal species, preferably human. Such vaccine may include any Orthopoxvirus, and the Orthopoxvirus may be live, attenuated or killed. Orthopoxvirus vaccine can also be a recombinant Orthopoxvirus, or a recombinant virus from any genus that is engineered to express an Orthopoxvirus antigen. The Orthopoxvirus vaccine may also be an Orthopoxvirus antigen isolated from an Orthopoxvirus or expressed by recombinant DNA technology. The Orthopoxvirus vaccine may also be DNA vaccine encoding an Orthopoxvirus antigen.
Orthopoxvirus neutralizing antibody—An Orthopoxvirus antibody or antibody fragment which specifically inhibits a member of the genus Orthopoxvirus from infecting a cell. Such neutralizing antibodies and fragments are typically detected using a cell culture assay.
Orthopoxvirus infection—an infection caused by a virus belonging to the Orthopoxvirus genus. Such infection may be the result of an adverse reaction to vaccination with vaccinia virus. Alternately, such an infection may be caused by naturally occurring or cultured virus belonging to the genus Orthopoxvirus, genetically engineered virus belonging to the genus Orthopoxvirus, or a virus from any genus having antigens recognized by Orthopoxvirus antibodies.
VIG—vaccinia immune globulin, a pharmaceutical immune globulin formulation of vaccinia virus antibodies produced by stimulating donors, typically human, with a vaccinia virus vaccine and fractionating the donor blood. Such VIG may be produced by anion exchange chromatography and be formulated for intravenous injection.
OPVIG—Orthopoxvirus immune globulin, a pharmaceutical immune globulin formulation of Orthopoxvirus antibodies produced by stimulating donors, preferably human, with an Orthopoxvirus vaccine and fractionating the donor blood.
OPVIF—Orthopoxvirus immune formulation, a pharmaceutical formulation of Orthopoxvirus antibodies, or fragments of antibodies.
Donor—an animal, preferably human, who supplies blood, plasma, serum, or any body fluid having an immune globulin.
IV—intravenous injection.
IM—intramuscular injection.
Virus binding antibodies—virus specific antibodies as detected by means known in the art, for example, an ELISA assay.
Vital Dye—as used herein means a dye that can be used to detect the vitality of a cell, or an enzyme introduced into the cell. Typically, such dyes are metabolized by enzymes to a substance with a different electromagnetic absorption spectrum than the vital dye.
   A Colorimetric Assay For Titration of Neutralizing Antibodies

Without being limited to a specific theory, it is generally believed that viral neutralizing antibodies bind to a virus and prevent it from infecting susceptible cells. The presently-accepted assay for the detection and quantification of neutralizing antibodies against vaccinia virus in an antibody solution such as blood, serum, or immune globulin preparation, is by the Plaque Reduction Neutralization Test Assay (PRNT). The PRNT assay involves incubating a constant dose of vaccinia virus with dilutions of the antibody solution at 37° C., prior to infecting cells in vitro. The infected cells are then plated onto cell culture plates overlaid with a semisolid medium. Cells infected with the virus are killed or their growth rate is inhibited. The virus, shed from the infected cells infects neighboring cells, causing their death or inhibition of growth. The result is a plaque or area of the cell culture plate with greatly reduced cell numbers, surrounded by areas of heavy cell density. Neutralizing antibody titre is then calculated by counting viral plaques formed at a given dilution of the antibody solution, and more rarely, light microscopic evaluation of the cytopathogenic effects caused by the virus.

The PRNT assay has many drawbacks. The assay is cumbersome and requires the manipulation of concentrated vaccinia virus stocks. The assay is labor intensive in that each sample to be assayed requires that plaques be counted from multiple cell culture plates. Counting the plaques is a very time consuming task, and the assay results may be subject to error introduced by the experience, qualifications and bias of the operator or person counting the plaques, and may therefore not be reproducible.

To overcome or ameliorate some of the limitations of the above PRNT assay, a colorimetric assay has been developed for detection of neutralizing antibodies against vaccinia virus. The colorimetric assay is based on the ability of neutralizing antibodies, if present in an antibody solution, to prevent or inhibit vaccinia virus from infecting and killing susceptible cells in culture. The fraction of cells infected or killed is then determined by exposing the cells to a dye, wherein metabolism of the dye is dependent on the numbers of living or infected cells.

In one embodiment of the invention, following infection, the cells are exposed to a vital that is water-soluble wherein living cells metabolize the dye. The colorimetric assay is performed by serially diluting an antibody solution, and incubating the dilutions with a given number of plaque-forming units (PFU) of the Lister strain of vaccinia virus, available from the ATCC (ATCC Catalogue No. VR-862). Neutralizing antibody, if present in the antibody solution, will bind to the vaccinia virus and inhibit it from infecting cells. The virus/antibody mixture is then transferred to a 96-well plate containing monolayers, of a cell type susceptible to infection by vaccinia virus, such as BS-C-1 cells (ATCC Catalogue No. CCL-26). Wells are also prepared for the two controls: uninfected cells to which no virus was added; and infected cells to which virus was added without any neutralizing antibody. After incubation for 3 to 4 days at 37° C., or until more than 80% of the control infected cultures exhibit high degree of cytopathologic effect, a vital dye is added. In one embodiment of the present invention, the dye is the tetrazolium salt WST-1 (4-(3-(4-Iodophenyl)-2-(4-nitrophenyl)-2H-5-tetrazolio)-1,3-benzene disulfonate) (Roche Molecular Biochemicals). Live cells in the culture reduce the WST-1 to its formazan derivative in a reaction catalyzed by mitochondrial dehydrogenase within the live cells. The reduction reaction of WST-1 is halted in dead cells. Therefore, the amount of WST-1 reduced to its formazan derivative is proportional to the amount of living cells in the culture. The formazan formed is detected by absorption between 420-480 nm by an automated microplate spectrophotometer. The 50% activity of the antibody solution is determined by the dilution required for protection of 50% of the cells from being killed as calculated by the formula:
50%×[optical-density reading of uninfected cells minus optical-density reading of control infected cells].

This colorimetric assay has the advantage that the results are read on a spectrophotometer, and therefore less susceptible to any bias introduced by an operator counting plaques. Furthermore, a standard curve can also be run using an antibody solution with known levels of neutralizing antibodies. This will allow for the assay to be standardized and for reproducible results, and allow for comparisons of results between assays.

As will be obvious to those skilled in the art, many different cultured cell types are susceptible to vaccinia virus infection, and may be used in the assay LLC-MK2 cells (ATCC CCL-7), Vero (ATCC No: CCL-81), HeLa cells (ATCC CCL-2); CV-1 cells (ATCC CCL-70). The ATCC lists the cell types that are most susceptable to infection for each cell type. Additionally other vaccinia virus strains may be used including the NYCBOH-Wyeth strain (ATCC Catalogue No. VR-1536) or IHD (ATCC VR-156). Also, many vital dyes are known and may be used to detect the amounts of living cells. Also, different strains of vaccinia virus are available may also be used in this assay.

The colorimetric assay may also detect Orthopoxvirus enzymes that are expressed in infected cells or enzymes that are introduced by recombinant Orthopoxviruses. In one embodiment, an orthopoxvirus may be engineered to express luciferase, which may be detected by adding the substrate luciferine and ATP.

The colorimetric assay can be used for a variety of uses. The plasma of Orthopoxvirus vaccine recipients may be assayed to determine whether the vaccine recipient has been properly immunized by determining the levels of neutralizing antibodies to vaccinia virus present in the blood of the vaccine recipient.

This colorimetric assay can be used to screen plasma from donors for high levels of neutralizing antibodies to vaccinia virus. That is, in some embodiments, plasma from donors having titers below a threshold level may be rejected and only plasma having above titers above a threshold level are pooled. Manufacture of VIG by fractionation of plasma with high levels of neutralizing antibodies to vaccinia virus may allow for the production of VIG with a higher potency. The assay can also be used to test the potency of VIG. The assay may also be used to evaluate different Orthopoxvirus vaccines to determine which vaccine elicites most effective Orthopoxvirus neutralizing antibodies. In such embodiments, it is important that the virus used can be neutralized, or prevented from infecting cells, by the antibodies in the solution being tested.

ELISA method for detection of vaccinia virus binding antibodies.

Antibodies that specifically bind the vaccinia virus can be detected using an enzyme linked immunosorbent assay (ELISA). The ELISA method is a direct-binding assay to which quantification has been introduced via the assignment of arbitrary units (volumetrically based) to a purified polyclonal vaccinia Immune Globulin formulation (VIG) produced by Baxter and available from the Centers for Disease Control and Prevention (Atlanta, Ga.). The Baxter VIG standard (Lot No. 0448A101AE) is the current reference material and has been assigned an arbitrary value of 10,000 U/mL. As of the date of filing this application, an FDA recognized standard for VIG was not established. Once an established standard is recognized, it may be obtained from Center for Biologics Evaluation and Research (CBER), Food and Drug Administration (Rockville, Md., USA). The assay is performed by coating a solid support, such as the wells of a microtiter plate, with a source of the desired target antigen (i.e. vaccinia viral lysate). To quantify the levels of antibodies which bind to the target antigen(s), a known volume of material suspected of containing vaccinia binding antibodies, such as serum, plasma or VIG product, is added to the microtitre wells. Vaccinia-binding antibodies, if present, will bind to the vaccinia antigens bound to the plate. The vaccinia binding antibodies are detected by addition of Anti-Human IgG antibodies conjugated to a reporter enzyme, such as Horse Radish Peroxidase (HRP). An enzyme substrate, which the reporter enzyme can convert to a colored product, is then added to the wells. The amount of colored product produced is directly proportional to the amount of vaccinia binding antibodies present in the sample and thus may be used as the basis of quantification.

As will be apparent to one of skill in the art, the substrates and enzymes used in the above example are for illustrative purposes and are by no means limited as other suitable enzyme and substrate combinations known in the art may be used.

Correlation Between ELISA and PRNT Assays

There are several technical advantages to an ELISA assay as compared to a PRNT assay. ELISA assays are faster in that the results are evident within a day, whereas PRNT assay may require 5 to 7 days to develop. Furthermore, many samples can be simultaneously tested in duplicate with an ELISA assay. The ELISA assay detects all vaccinia virus binding antibodies, not just the vaccinia neutralizing antibodies. Previously, it was believed that the results of an ELISA assay did not correlate to the results obtained by a PRNT assay in detecting vaccinia neutralizing antibodies. Lublin-Tennenbaum, T., et al (Viral Immun. 3:1 (1990) 19-25) describe a weak correlation coefficient of only 0.562 between results obtained by a PRNT assay and an ELISA assay for plasma obtained 3 weeks post vaccination (1.0 being perfect correlation and 0 being no correlation). Furthermore, these investigators state that there is no correlation for samples with low antibody levels. As such, to accurately select plasma with high levels of vaccinia antibodies required each sample be tested by the cumbersome PRNT assay.

An important finding in the present invention is the determination that the results of an ELISA assay, performed as described herein, is as sensitive as the PRNT assay. The inventors have determined that there is a correlation coefficient of 0.98 between the results obtained with an ELISA and those obtained using the PRNT assay. This discovery has significant implications as the level of vaccinia antibodies in plasma isolated from donors and plasma pools can be accurately determined by an ELISA assay.

Requirement to Vaccinate Plasma Donors

It is known that a naïve patient upon exposure to an antigen will produce an immune reaction in which the majority of immune globulin produced is IgM, and very little IgG. Subsequent exposure to the antigen will result in an immune reaction in which the majority of immune globulin produced is IgG. In one embodiment of the present invention, the plasma is fractionated to preferentially isolate IgG. In such an embodiment, donors having had previous exposure to vaccinia virus will be preferable.

Studies undertaken in this investigation have determined that the levels of vaccinia antibodies in the North American population are low. Plasma isolated from donors that had a medical history of Smallpox vaccination, but were not recently vaccinated for Smallpox. The levels of vaccinia antibodies in such donor plasma were very low and such plasma could not be formulated into a potent VIG product.

It is well known that immune globulin can be fractionated from plasma or serum obtained from selected donors who have significantly higher titers for a specific antibody than is normally found in the average population (U.S. Pat. No. 4,717,564 incorporated herein by reference). These donors have either been recently immunized with a particular vaccine (U.S. Pat. No. 4,174,388) or else they have recently recovered from an infection or disease [Stiehm, Pediatrics, Vol. 63, No. 1, 301-319 (1979)].

One embodiment of the present invention, donors who have been previously immunized with a vaccinia virus vaccine will be challenged with Dryvax®. Plasma isolated from such donors will be screened for the presence of vaccinia virus antibodies by ELISA assay. Plasma with significantly higher levels of vaccinia antibodies compared to the non-stimulated donors will then be processed into a VIG product.

Those skilled in the art will understand that the plasma donors can be vaccinated with other strains of vaccinia virus vaccine or an Orthopoxvirus vaccine. As will be obvious to those skilled in the art, the donor plasma may be screened for the presence of for vaccinia antibodies by alternate methods such as the PRNT assay or a colorimetric assay.

In alternate embodiments of the present invention, the donor may be a naïve donor who is immunized with an vaccinia virus vaccine or Orthopoxvirus vaccine for the first time prior to donation or blood or plasma.

Orthopoxvirus Vaccine

It is known that the major antigens are shared between viruses within the Orthopoxvirus genus, and that antibodies produced against vaccinia virus will protect against infections of variola virus, cowpox and monkey pox virus. Therefore, donors may be vaccinated with viruses belonging to the Orthopoxvirus genus, or antigens from viruses belonging to the Orthopoxvirus genus, to stimulate the production of antibodies against the variola virus. Likewise, such antibodies generated against one virus belonging to the Orthopoxvirus genus may be protective against other viruses belonging to the Orthopoxvirus genus. The term Orthopoxvirus vaccine will therefore be used to indicate Orthopoxvirus antigen capable of stimulating the production of an immune globulin specific to a virus belonging to the Orthopoxvirus genus.

The currently available vaccinia (Smallpox) vaccine is Dryvax®, and is obtained from the Centers for Disease Control and Prevention in Atlanta, Ga. Dryvax® is a lyophilized, live virus preparation of infectious vaccinia virus, specifically the NYCBOH strain of vaccinia virus. It does not contain Smallpox virus (variola).

Epidemiological studies demonstrate that an increased level of protection against Smallpox persists for <5 years after primary vaccination and substantial but waning immunity can persist for >10 years. Antibody levels after revaccination can remain high longer, conferring a greater period of immunity than occurs after primary vaccination alone. An important discovery of the present invention was the discovery that revaccination of individuals with the Smallpox vaccine is safe, even though the primary vaccination occurred more than 30 years ago. Another important discovery was that revaccination with Smallpox vaccine more than 30 years after primary vaccination elicited a strong IgG antibody response.

As will be obvious to those skilled in the art, an Orthopoxvirus vaccine may also be generated from vaccinia virus strains other than the NYCBOH strain used in the Dryvax® vaccine. The important factor is that the vaccinia virus strain be capable of eliciting neutralizing Orthopoxvirus antibodies in humans. The smallpox eradication campaign used vaccines derived from many vacinia virus strains. In the United States, these included the New York City calf lymph (NYC_CL) and New York City chorioallantoic membrane (NYC_CAM) strains, both of which were derived from a seed virus of the NYCBOH strain. Other strains used frequently in the global eradication program were EM-63 (USSR) and Temple of Heaven (China). The Lister or Elstree (United Kingdom) strain, prepared on the skin of sheep, was used extensively in Europe and other parts of the world. The Lister strain was distributed by the WHO International Reference Centre to production laboratories for use as seed lots, and from 1968 to 1971, the Lister strain became the most widely used throughout the world.

Because the major antigens are shared between viruses within the Orthopoxvirus genus, other Orthopoxviruses may be used as an Orthopoxvirus vaccine to immunize donors. For example, the donors may be immunized with a strain of cowpox virus that may have low virulence in humans and elicit neutralizing Orthopoxvirus antibodies in humans. The Orthopoxvirus vaccine may also be an Orthopoxvirus attenuated by, for example, irradiation, heat, chemical treatment, and other methods known in the art. Attenuation may also be achieved by reducing the virulence of the virus through removal of certain genes or by other genetic manipulation to modify gene expression. The Orthopoxvirus vaccine may also be a recombinant virus having low virulence in humans.

Recombinant vaccinia viruses have been created from different strains of vaccinia virus. Examples of such recombinant viruses are described in U.S. Pat. No. 6,177,076 titled “Method of treating bladder cancer with wild type vaccinia virus”, U.S. Pat. No. 6,093,700 titled “Method of inducing an immune response using vaccinia virus recombinants encoding GM-CSF” and U.S. Pat. No. 5,656,465 titled “Methods of in vivo gene delivery”. In the United States, recombinants have been made from a non-attenuated NYCBOH strain, or a mouse neuroadapted derivative, the WR strain. Recombinants have also been made using the Copenhagen and Lister vaccinia strains, which are more pathogenic among animals than the NYCBOH strain. Additionally, certain highly attenuated, host-restricted, non- or poorly replicating poxvirus strains have been developed for use as substrates in recombinant vaccine development. These strains include the Orthopoxviruses, modified vaccinia Ankara (MVA) and NYVAC (derived from the Copenhagen vaccinia strain), and the Avipoxviruses, ALVAC and TROVAC (derived from canarypox and fowlpox viruses, respectively) (Paoletti E, et al. Dev Biol Stand 1995;84:159-63; Perkus M E, et al. Ann N.Y. Acad Sci 1995;754:222-33; Sutter G, Moss B. Dev Biol Stand 1995;84:195-200.).

Animal studies indicate that recombinants are usually less pathogenic than the parent strain of vaccinia virus (Lee M S, et al. J Virol 1992;66:2617-30.). Laboratory-acquired infections with non-highly attenuated vaccinia and recombinant viruses derived from non-highly attenuated vaccinia strains have been reported (Pike R M. Health Lab Sci 1976; 102:105-14; Jones L, et al, Nature 1986;319:543; Shimojo J. Bibi Haematol 1975;40:771-3; Openshaw P J M, et al. Lancet 1991-338:459.). However, highly attenuated poxvirus strains (MVA, NYVAC, ALVAC, and TROVAC) are unable to replicate (MVA, ALVAC, and TROVAC) or replicate poorly (NYVAC) in animal host cells; therefore, highly attenuated poxvirus strains do not create productive infections (Moss B. Dev Biol Stand 1994;82:55-63.). These highly attenuated strains have also been reported to be avirulent among normal and immunosuppressed animals (MVA, NYVAC, ALVAC, or TROVAC) and safe among humans (MVA) (Paoletti E, et al. Dev Biol Stand 1995;84-.159-63; Sutter G, Moss B. Dev Biol Stand 1995;84-.195-200; Tartaglia J, et al. AIDS Res Hum Retroviruses 1992;8:1445-7.). Such strains may be used as Orthopoxvirus vaccines to stimulate the production of Orthopoxvirus antibodies.

An Orthopoxvirus vaccine may also consist of an Orthopoxvirus antigen isolated from an Orthopoxvirus. Alternatively, the genes encoding the vaccinia virus immunodominant antigen have been isolated (Chertov O Yu, et al, Biomed Sci (1991) 2:151-4, Demkowicz W E, et al, J Virol (1992) 66:386-98). To generate an Orthopoxvirus vaccine, the genes encoding such immunodominant antigens may be expressed using recombinant DNA technology and be combined with adjuvant and administered into plasma donors. The Orthopoxvirus vaccine may consist of DNA vaccines, wherein genes encoding the immunodominant Orthopoxvirus antigens are cloned into appropriate DNA vectors and introduced into the plasma donors in such a manner that the viral antigen is expressed and elicits an immune reaction. The Orthopoxvirus vaccine may consist of recombinant non-Orthopoxvirus virus species that are engineered to express an Orthopoxvirus antigen. One virus that is used as such a vector is the vesicular stomatitis virus or VSV. The Orthopoxvirus vaccine may be a microorganism, for example, a recombinant microorganism, that expresses the Orthopoxvirus antigens and is capable of eliciting an immune response.

As will be evident to those skilled in the art, an Orthopoxvirus vaccine, used to immunize an animal, will be administered in such a manner that the Orthopoxvirus vaccine elicits an immune response in said animal. Routes of administration include dermal, inhaled, intraperitoneal, intramuscular, intraocular, intracranial or subcutaneous injections. Such Orthopoxvirus vaccines may be administered with or without an adjuvant such as Freund's complete or incomplete adjuvant.

Generation of Orthopoxvirus Antibodies

The term Orthopoxvirus antibody is used herein to describe antibodies raised against one virus belonging to the Orthopoxvirus genus, which may recognize and bind to another virus belonging to the Orthopoxvirus genus. Orthopoxvirus antibodies used in the present invention may be polyclonal antibodies specific for Orthopoxvirus antigens generated using conventional procedures in humans and animals, as described above. Following several booster immunizations, samples of serum or plasma are collected and tested for reactivity to the Orthopoxvirus antigen, or neutralizing antibody against vaccinia virus in standard assays, examples of which are described above. Particularly preferred polyclonal antisera will give a signal on one of the assays that is greater than background. Once the blood antibody titer of the animal has reached a plateau in terms of its reactivity to the antigen, larger quantities of antisera may be readily obtained either by periodic (e.g. weekly) plasmapheresis, withdrawing whole blood and isolating plasma, or by exsanguinating the animal and isolating plasma or serum from the blood thus collected.

Human Orthopoxvirus antibodies may be produced in human volunteers. For example, an Orthopoxvirus antibody may be obtained from a subject previously immunized with an Orthopoxvirus vaccine such as Dryvax®, and given booster immunizations of Orthopoxvirus vaccine. Alternatively, an Orthopoxvirus antibody may be obtained from a subject initially immunized naturally with an Orthopoxvirus infection, and given booster immunizations of Orthopoxvirus vaccine.

Monoclonal Orthopoxvirus antibodies may also be readily generated using conventional techniques (see U.S. Pat. Nos. RE 32,011, 4,902,614, 4,543,439, and 4,411,993 which are incorporated herein by reference; see also Monoclonal Antibodies, Hybridomas: A New Dimension in Biological Analyses, Plenum Press, Kennett, McKearn, and Bechtol (eds.), 1980, and Antibodies: A Laboratory Manual, Harlow and Lane (eds.), Cold Spring Harbor Laboratory Press, 1988, which are also incorporated herein by reference).

Other techniques may also be utilized to construct monoclonal antibodies (see William D. Huse et al., “Generation of a Large Combinational Library of the Immunoglobulin Repertoire in Phage Lambda,” Science 246:1275-1281, December 1989; see also L. Sastry et al., “Cloning of the Immunological Repertoire in Escherichia coli for Generation of Monoclonal Catalytic Antibodies: Construction of a Heavy Chain Variable Region-Specific cDNA Library,” Proc Natl. Acad. Sci USA 86-.5728-5732, August 1989; see also Michelle Alting-Mees et al., “Monoclonal Antibody Expression Libraries: A Rapid Alternative to Hybridomas,” Strategies in Molecular Biology 3:1-9, January 1990; these references describe a commercial system available from Stratacyte, La Jolla, Calif., which enables the production of antibodies through recombinant techniques). Similarly, binding partners may also be constructed utilizing recombinant DNA techniques to incorporate the variable regions of a gene that encodes a specific binding antibody (See Bird et al., Science 242:423-426, 1988).

It will be apparent to one skilled in the art that the immune globulin preparations used in the present invention may contain more than one type of Orthopoxvirus antibody. For example, donors may be vaccinated with antigens from various difference members of the Orthopoxvirus family. The plasma from the various donors may be combined and fractionated to produce an immune globulin preparation that contains antibodies specific to several members of the Orthopoxvirus family. Alternatively, immune globulin fractionated from the plasma of donors vaccinated with antigens from various difference members of the Orthopoxvirus family may be combined into one immune globulin product.

Isolation of Plasma

Plasmaphoresis involves the removal of the cellular components of whole blood and isolation of the plasma. Typically, such cellular components of whole blood are returned to the patient. Plasma may be collected by an automated plasmaphoresis device such as the PCS®2 System by Haemonetics Corporation (Braintree, Mass.) or the Autopheresis-C from Baxter (Round Lake, Ill.). Essentially, a hypodermic needle is inserted into a vein in the donor's arm. The plasmaphoresis device then cycles through a blood withdrawal phase, plasma separation and collection phase, and reinfusion phase. A volume of blood is withdrawn and mixed with an anticoagulant solution, such as sodium citrate. This anticoagulated blood is pumped through a separation device that separates the cellular components from the plasma. The plasma is retained in a collection container while the cellular components are pumped back to the donor. Typically 600 to 700 mls of plasma are removed from a donor every plasmaphoresis session and a donor may undergo plasmaphoresis two to three times a week.

As will be obvious to those skilled in the art, alternate methods may also be employed to isolate the plasma including isolating whole blood from a patient, adding an anticoagulant, centrifuging the whole blood and removing the overlaying plasma layer. The cellular component can then be returned to the patient.

Screening of Donor Plasma

The plasma donors are screened for general good health and any lifestyle habits, such as drug use. Potential donors, and donor plasma, is screened for variety of known or emerging pathogens, and plasma that is positive for such pathogen markers is rejected. The testing includes, but is not limited to, Hepatitis A, Hepatitis B, Hepatitis C, HIV-1, HIV-1, HTLV-1, HTLV-2. Such tests are typically for antibodies to such pathogens, but may also include tests for specific antigens that may be present. Further testing may include PCR tests for DNA or RNA from certain viral pathogens as described herein below. Such tests are typically performed by commercial medical testing facilities or medical laboratories. The VIG product is also screened for a variety of know and emerging pathogens by antibody and PCR analysis.

Following vaccination, the donor plasma is screened for the presence of vaccinia antibodies by an ELISA method. Donor plasma with antibody levels below the threshold is rejected. In one embodiment, the threshold limit for vaccinia antibodies is 200 U/ml. Donor plasma with antibody titres above the threshold is pooled and fractionated to isolate the immune globulin.

The final immune globulin product should have a protein level within acceptable levels that will allow the product to be injected intravenously in a reasonable volume. The threshold level for antibody in donor plasma is a level that will dictate the potency of the final immune globulin product (i.e. the units of vaccinia antibodies per unit volume). As will be apparent to one of skill in the art, achieving a more potent final immune globulin requires selecting donor plasma with a higher level of antibodies.

Other methods may also be used to screen the donor plasma including the PRNT assay and the colorimetric assay. In the event another Orthopoxvirus vaccine is used to vaccinate the donors, the ELISA method may require modification, such as binding the. Orthopoxvirus vaccine to the ELISA plate to accurately assay the levels of Orthopoxvirus specific antibodies.

Previous studies have shown that the levels of vaccinia antibodies in plasma from donors previously vaccinated with the Lister (Elstree) strain of vaccinia virus leak at about 14 days post vaccination, and decrease to near normal levels over the next 60 days (Stienlauf S., et al Vaccine 17 (1999) 201-204). The inventors have shown that the same antibody response curve is realized for vaccination with the NYCBOH (Dryvax®) strain of vaccinia virus.

PCR Testing Donor Plasma and Product for Vaccinia DNA

One important aspect of the present invention is the test of the plasma pools and the final VIG product for the presence of vaccinia virus and vaccinia DNA. As the plasma donors are vaccinated with a live virus, the possibility exists that vaccinia virus can enter the blood circulation of the donor and be isolated in the plasma. Furthermore, the vaccinia virus may then be found in the VIG product thus formulated. The VIG product is used to treat individuals with an Orthopoxvirus infection or prophylactically if the individual believes or expects to be exposed to an Orthopoxvirus. In such individuals, intravenous injection of vaccinia virus together with the VIG may result in a severe infection or exacerbate an existing infection.

To detect vaccinia virus in donor plasma, or vaccinia virus in the final VIG product, the samples were tested for the presence of vaccinia DNA by PCR. If virus is present in the sample, the virus must first be disrupted to release the DNA. The sample may be treated with QIAamp® UltraSens™ Virus Kit (Qiagen) according to instructions provided by the manufacturer. The vaccinia viral DNA, if present, may be detected by methods known in the art and described in U.S. Pat. Nos. 5,780,222, 5,176,995 and WIPO Application No 9322456. As will be appreciated by those skilled in the art, the sequence of the forward and reverse primers for PCR or Polymerase Chain Reaction will be designed to amplify a sequence in the vaccinia genome. Knowing the sequence of the vaccinia virus genome, primers may be designed using software such as Primer Express software version 2.0 from Applied Biosystems.

Preferably, the detection method chosen will ensure that the numbers of vaccinia viral genomes in a sample of plasma or in a sample of the final VIG product are below a certain threshold value. One method is by quantitative PCR (QPCR) (Mackay, et al, (2002) Nucleic Acids Res., 30, 1292-1305; Lovatt, A. (2002) Rev. Mol. Biotechnol., 82, 279-300). Essentially, a forward and reverse primer set is designed to amplify a part of the viral genome. An oligonucleotide target is designed that anneals to a sequence that is between the primer set, and that will be amplified. In one embodiment of the invention, such an oligonucleotide target includes a covalently attached fluorescent molecule, an oligonucleotide and a covalently attached fluorescence quencher such that the fluorescence quencher inhibits the fluorescent molecule from fluorescing in the oligonucleotide. The covalent bond between the oligonucleotide and the fluorescent molecule and/or the fluorescence quencher is susceptible to cleavage by a thermostable DNA polymerase such as Taq.

In operation, DNA is extracted from the sample, and the sample is subjected to thermocycling in the presence of the primer set, oligonucleotide target, Taq enzyme, nucleotides and other such elements required for a polymerase chain reaction to occur and amplify the part of the viral genome. If vaccinia virus DNA is present in a sample, the oligonucleotide target will anneal to the viral genome between the primer set. As the Taq enzyme amplifies part of the viral genome, the Taq will degrade the annealed oligonucleotide target, thereby releasing the fluorescent molecule and/or the fluorescent quencher and resulting in an increase in the amount of fluorescence. The fluorescence may be quantified on an ABI PRISM® 7700 Sequence Detection System (Applied Biosystems, Foster City, Calif.). A standard curve is established by performing the above-described QPCR on plasma to which known numbers of vaccinia viruses are added. The threshold cycle values (Cₜ) copy numbers are determined (number of cycles required for the fluorescent signal to reach a specific level). The standard curve is a plot of the Cₜ values versus the copy number of virus. (The Cₜ values are inversely proportional to the starting copy number.) The copy number of vaccinia virus in the sample is determined by interpolation of the standard curve. Analysis of donor plasma and VIG product have shown that there is less than 0.13 pg of vaccinia DNA or less than 1250 copies of Vaccinia DNA per ml.

Fractionation of Plasma to Isolate Immune Globulins

In one embodiment of the present invention, a human immune globulin fraction containing IgG, suitable for intravenous injection, is prepared from human donor plasma by contacting an aqueous donor plasma fraction containing IgG with one or more chromatographic separation columns to produce a purified immune globulin fraction. The immune globulin preferably is at least about 95% (w/w) pure, more preferably about 99.5% pure. To be suitable for intravenous administration, the final immune globulin product should not contain impurities, such as IgG polymers and/or fragments, which possess complement fixing activities. Preferably the final immune globulin contains at least 94% monomeric IgG, as determined by HPLC analysis as outline in the European Pharmacopoeia 4th Edition.

In one embodiment, donors are immunized with a vaccinia virus vaccine and plasma is selected for the presence of binding or neutralizing antibodies to vaccinia virus. The plasma is modified to the ionic strength and pH of the initial buffer used with the chromatographic separation column. In an embodiment of the invention, the aqueous animal plasma fraction is contacted with one or more, preferably one or two anionic exchangers to produce a purified IgG rich fraction.

The purified IgG rich fraction may optionally be treated with a solvent and detergent to inactivate lipid enveloped viruses. Suitable solvents and detergents which may be used to inactivate lipid envelope viruses include Triton X-100 and tri (n-butyl) phosphate. The solvents and detergent may be removed by conventional methods such as reverse phase chromatography. The purified IgG fraction may be further concentrated using ultrafiltration.

The chromatographic separations may be carried out on anion exchangers using the procedure as described in Canadian Patent Nos. 1,168,152 and 1,201,063, which are incorporated herein in their entirety by reference. By way of example, the aqueous animal plasma fraction is applied to an anion exchange column that may contain an agarose cross-linked anionic exchange resin such as DEAE-Sepharose™ CL6B or DEAE-Biogel™, and an IgG rich fraction is obtained by elution with an equilibrating buffer. The IgG rich fraction may be concentrated for example by ultrafiltration. The IgG rich fraction is applied to a second different anion exchange column such as DEAE-Biogel™, or DEAE-SephadeX™-50. A purified IgG rich fraction is isolated by elution with an appropriate equilibrating buffer. For immune globulin formulations fractionated by ion exchange chromatography, the immune globulin is not subjected to chemical or enzymatic modification to reduce aggregate concentrations or compliment activity.

Other methods for fractionating plasma to obtain an immune globulin fraction are known and may also employed. For example, one may employ the Cohn fractionation method (referenced herein below, which references are incorporated herein by reference thereto), an ammonium sulphate fractionation, polyethylene glycol precipitation or the like. The aforementioned immune globulin comprises IgG, usually at least 90% IgG monomer. The material generally also contains other globulins such as IgA, IgM, and the like. Such fractionated plasma may be further subjected to ion exchange chromatography to reduce or remove the IgA and IgM.

The plasma may also be subjected to Cohn fractionation to produce Fraction II (Cohn et al, J. Am. Chem. Soc., 68, 459 (1946) and Oncley et al, ibid., 71, 541 (1949)). The Fraction II material must be further purified before formulation into an intravenously administrable product, and this can be accomplished via any of a number of known procedures so that the final immune serum globulin product does not contain impurities, such as IgG aggregates and/or antibody fragments, that possess complement activating activities. For example, to disaggregate the IgG isolated by Cohn precipitation, the IgG may be treated with a protease such as pepsin, or an acidic pH.

An embodiment of the present invention relates to the production of an immune globulin preparation composed mainly of IgG1, IgG2 and IgG3, but deficient in IgG4, and having low concentrations of IgA. Such an immune globulin preparation may be prepared by the above described ion exchange chromatography process as detailed in Canadian Patent Nos. 1,168,152 and 1,201,063, and by one or more of the processes described above. Immune globulin preparations manufactured according to an alcohol precipitation method such as the Cohn method, referenced above, contain all four IgG subtypes as well as higher levels of IgA. In certain applications, an immune globulin preparation containing IgG4 and IgA may be disadvantageous. For example, a fraction of human patients are deficient for the secretion of IgA. Repeated administrations of immune globulin preparations having significant levels of IgA can result in an anaphylactic reaction in the individual which may be fatal. The immune globulin subtype IgG4 is primarily associated with allergies, and its presence in immune globulin preparations may be deleterious if high levels are injected into certain patients.

Although the fractionation methods described above refer to the fractionation of plasma, other blood fractions may also be used. For example, serum, or other blood fractions may be fractionated. Other methods of isolating immune globulin may also be used to isolate other classes of immune globulins, such as IgA, IgM or IgD.

As will be evident to those skilled in the art, the above described fractionation process may also be used on plasma from donors vaccinated with an Orthopoxvirus vaccine. The above fractionation process may also be used to prepare immune globulin formulations suitable for administration by various routes, such as intramuscular, intranasal, subcutaneous, oral, enteral or parenteral use.

Formulations of VIG

Purified immune globulin fractions that may be formulated into pharmaceutical compositions for administration into humans are well known in the art. A pharmaceutical composition of purified immune globulin fraction from a donor vaccinated with vaccinia virus vaccine is referred to as vaccinia immuneglobulin or VIG.

The purified immune globulin fraction may be formulated with a surfactant such as Polysorbate 80, also known as Tween 80, at a concentration from 0.01-0.5% (w/v), preferably 0.03%. The surfactant helps to reduce aggregation over time with the liquid protein solution at the glass/liquid/air interface to provide a highly stable preparation enriched for Orthopoxvirus antibodies. The presence of a surfactant in an immune globulin formulation has also been shown to increase the serum half life of the immune globulin (WO9844948).

The purified immune globulin rich fraction obtained using the process described above may be further stabilized by the addition of stabilizers such as mannitol, glycine (e.g. 0.1 M glycine), and sodium chloride (e.g. 0.15 M sodium chloride), and the pH of the fraction may be adjusted within the range 4.0 to 7.6. The resulting preparation may be sterilized for example, by filtration, and it may be used in this form. If desired the preparation may be freeze-dried, and reconstituted using a suitable solution, e.g. 0.9% sodium chloride.

In one embodiment, a preparation obtained using the process described above has the following characteristics: 2-3% human immune globulin, no or very low level buffer, essentially no ionic strength, 0.03% Polysorbate 80, 10% sorbitol, pH 4.0.

The purified immune globulin rich fraction may also be formulated into a liquid formulation. Maltose may be added to the immune globulin solution to a final concentration of 8-12% maltose. Polysorbate 80 may be added to a final concentration of 0.03%. The resulting liquid immune globulin formulation may be stored at 4° C. for extended periods of time.

The compositions described herein can be prepared by methods known for the preparation of pharmaceutically acceptable compositions which can be administered to patients, such that an effective quantity of the active substance is combined in a mixture with a pharmaceutically acceptable vehicle. Examples of intravenously-administrable immune globulin preparations are described in U.S. Pat. No. 5,945,098, incorporated herein by reference. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the vaccinia virus antibodies in association with one or more pharmaceutically acceptable vehicles or diluents, and contained in buffered solutions with a suitable pH and iso-osmotic with the physiological fluids.

Pharmaceutical techniques may also be employed to control the duration of action of the compositions/preparations of the invention. Control release preparations may be prepared through the use of polymers to complex, encapsulate, or absorb the vaccinia virus antibodies.

As will be evident to those skilled in the art, the above described formulation process may also be used on immune globulin obtained from donors vaccinated with an Orthopoxvirus vaccine. Such a pharmaceutical formulation is referred to as Orthopoxvirus immune globulin (OPVIG). As described above, Orthopoxvirus antibodies may also be monoclonal antibodies or fragments thereof produced in cell culture, or antibodies or fragments thereof produced in microorganisms by recombinant DNA technology. Such Orthopoxvirus antibodies may also be formulated into pharmaceutical compositions, and are referred to as Orthopoxvirus immune formulations (OPVIF).

IV Versus IM Administration of Immune Globulin Formulations to Human Patients

An embodiment of the present invention is a pharmaceutical immune globulin formulation suitable for intravenous injection or infusion to maximize bioavailability, reduce dosage, and to elicit faster pharmacodynamic action i.e. neutralization of circulating Orthopoxvirus. Evidence exists that the bioavalability of an immune globulin formulation administered IV is far greater than when administered IM. A clinical study was performed with an immune globulin formulation WinRho SDF™ containing antibodies to the Rh factor (anti-D antibodies). The levels of anti-D antibodies were determined following administration of 300 μg of WinRho SDF™ (obtained from Cangene Corporation, Winnipeg, Manitoba, Canada) either intramuscularly or intravenously. For immune globulin administered intravenously, anti-D immune globulin levels in the circulation peaked one hour post injection, compared to the IM administration where the levels peaked 48 hours after injection. Furthermore, the highest concentration of anti-D immune globulin detected after IV administration was 125.5 ng/ml, compared to only 63.7 ng/ml after IM injection.

The above results indicate that administration of an immune globulin via intravenous administration results in much higher levels of immune globulin appearing in the circulation at a much earlier time as compared to intramuscular injection. In a patient suffering from an Orthopoxvirus infection, it may be very beneficial to administer an immune globulin formulation containing viral binding or viral neutralizing antibodies such that high levels of the immune globulin appear rapidly in the circulation. The above results indicate that a VIG, formulated for use via intravenous administration, may have considerable advantages in treating an Orthopoxvirus infection as compared to a VIG-IM, which can only be used for intramuscular administration.

Additionally an IV injection offers less discomfort to the patient as compared to an IM injection. The IV route of administration allows larger volumes of the VIG to be administered as compared to an IM route of administration. The IV route of administration is also preferable in very young children or elderly patients who may have limited amount of muscle mass.

Administration of VIG, IPVIG and OPVIF to Human Patients

Compositions/preparations of the invention contain Orthopoxvirus antibodies, either alone or together with an Orthopoxvirus vaccine. Such compositions are for intravenous, intranasal, intramuscular, subcutaneous, oral, enteral or parenteral use. In particular, those forms for intramuscular or subcutaneous administration are used, or forms for infusion or intravenous injection are used, which can be prepared as solutions of the antibodies or as powders of the antibodies to be mixed with one or more pharmaceutically acceptable excipients or diluents, suitable for the aforesaid uses and with an osmolarity which is compatible with the physiological fluids. For example, as described herein, an Rh antibody preparation may be formulated with a wetting agent and/or stabilized by addition of a stabilizer. When administering the compositions/preparations of the invention by injection, the administration may be by continuous infusion, or by single or multiple boluses.

Dosing Regimen

In one embodiment, the invention provides a method for treating or preventing an Orthopoxvirus infection comprising administering an effective amount of VIG to a patient in need thereof. An “effective amount of VIG” means that the amount of VIG administered to a patient with an Orthopoxvirus infection that is sufficient to treat or prevent an infection with an Orthopoxvirus.

For VIG-IM (available for the Centers for Disease Control and Prevention in Atlanta, Ga.) manufactured by a Cohn fractionation method and having a 16 g % (w/v) protein content, the recommended dose is outlined below.

Dosing Regimen for Intramuscular VIG

(a) Smallpox—Prevention or Modification

A dose of 0.3 mL/kg of body weight should be given within 24 hours of exposure. Exposed individuals should be simultaneously vaccinated or revaccinated with Smallpox vaccine unless otherwise contraindicated.
   (b) Vaccinia Infections—Prevention or Modification
A dose of 0.3 mL/kg of body weight should be given simultaneously with Smallpox vaccination. In cases of accidental exposure to vaccinia virus, this dosage should be given as soon as possible after exposure has occurred.
   (c) Treatment of Postvaccinal Complications
A dose of 0.6 mL/kg of body weight should be administered as soon as possible after symptoms appear. This dose may be repeated depending upon the severity of symptoms and response to treatment.
   (d) Post-Exposure Prevention or Modification of Smallpox
A dose of 0.3-0.6 mL/kg of body weight should be administered as soon as possible after confirmation or suspicion of inhalation exposure. However, such a dose has not been verified by rigorous scientific study.

As described herein below, a mouse tail pox model was used to demonstrate the efficacy of the VIG-IV product in treating a vaccinia infection. The VIG-IV formulation proved effective at doses from 3,000 U/Kg to 30,000 U/Kg in the mice. There was little difference in the efficacy of VIG IV in treating a mouse vaccinia infection at doses between the 30,000 U/Kg dose and the 3,000 U/Kg dose. This suggests that doses less than 3,000 U/Kg of VIG would continue to be effective against an Orthopoxvirus infection. Attempts have been made to determine the minimum protective level of antibody to Smallpox (Sarkar J K, et al, Bull World Health Organ (1975) 52:307-11). However, such studies were performed on a VIG formulated and injected IM, and may not be comparable to an IV VIG. A dose ranging study may be performed to determine the levels of vaccinia virus antibodies present in the circulation following administration of a given dose of VIG by IV injection. Based on such studies, the dose of VIG to be administered may be adjusted, in one embodiment lowered, from the above recommended doses.

Clinical Studies

Safety Studies

The pharmacokinetics and the safety of Vaccinia Immune Globulin (VIG), manufactured substantially as describe above, may be assessed by a clinical trial as described below. Such a clinical trial can be performed by administering two doses (0.3 mL/kg or 0.6 mL/kg) intravenously into normal healthy volunteers. Plasma samples are drawn over 42 days and tested for vaccinia IgG levels using the ELISA assay. Safety data is then collected by monitoring the volunteers.

A baseline analysis includes a brief physical, hematology, chemistry and urinalysis. Sampling IgG level time-points: Time 0, 30 min, 1 hr, 2 hr, 4 hr, 8 hr, 24 hr, Day 1 (same as 24 hr), 3, 5, 7, 14, 21, 28, 42. Plasma vaccinia immune globulin levels and derived pharmacokinetic parameters, vital signs, hematology (rbc, Hgb, Hct, wbc, differential cell counts, platelet count, rbc indices, prothrombin time, partial thromboplastin time, reticulocyte, fibrinogen) blood chemistry calcium, urea nitrogen, glucose [random], total bilirubin, AST, ALT, alkaline phosphatase, lactate dehydrogenase, creatinine, sodium, potassium, chloride, albumin, total proteins, urinalysis (appearance and color, S.G., protein, glucose, pH, occult blood, microscopic exam), adverse events.

The above data from the study are then collected and compiled, and the results compared between the two study groups to determine if the administration of the high does of VIG has any deleterious effects to the human subjects. As will be obvious to those skilled in the art, the above study may also be performed to test the safety of other Orthopoxvirus antibody formulations, such as OPVIG and OPVIF. The above study may also be used to test the safety of vaccinia virus or Orthopoxvirus antibody formulation administered via routes other than intravenous injection. As will be apparent to those skilled in the art, similar studies can be performed to determine the effective dose of an OPVIG and an OPVIF.

Test For Minimum Dose and Provide Evidence of Effectiveness of VIG Administered Prophylactically

After primary vaccination, neutralizing antibodies develop about the tenth day. Since this immune response occurs about four to eight days earlier than that which occurs with naturally acquired Smallpox, primary vaccination after exposure can sometimes curb or prevent Smallpox disease [Henderson, D. A., and B. Moss. Smallpox and Vaccinia. In Vaccines (S. A. Plotkin and W. A. Orenstein, eds), 1999 p. 74-97.]. One proposed protocol is the use of VIG to treat subjects exposed to Smallpox as well as to treat individuals who develop the moderate to severe adverse reactions to vaccinia vaccination. This protocol is based on the premise that if antibodies could be supplied to individuals known to have been exposed to virus before the secondary massive viremia occurs, the severity of the disease, which directly relates to the quantity of free virus (viral load) liberated in the blood, may be modified or perhaps aborted. The specific antibodies contained in vaccinia immune globulin would neutralize free circulating virus, diminish or suppress viremia, and thereby diminish or suppress infection of the mucous membranes and skin (Kempe, Henry C: Pediatrics, August 1960, pp 179).

A goal of the above study is to determine if administration of VIG in conjunction with or 3.5 days prior to vaccination with Dryvax® will attenuate or diminish the antibody response to vaccinia vaccination. Another goal of this study is to determine if the administration of VIG in conjunction with or 3.5 days prior to vaccination with Dryvax® will attenuate or diminish the local response to vaccinia vaccination at the vaccination site. Groups of naïve volunteers (i.e. volunteers that have not previously been vaccinated with vaccinia virus) receive either a placebo and vaccinia vaccine; doses of VIG 6,000 U/Kg and less (administered intravenously) on the same day as vaccinia vaccine; or doses of VIG 6,000 U/Kg and less (administered intravenously) four days prior to receiving vaccinia vaccine. The local skin reaction to vaccination is then examined and photographed, and the size measurement on day 7, 14, 21, 28, 42, 84. Results from the groups will then be compared to demonstrate attenuation of response to vaccination with concomitant or prior VIG administration. The levels of vaccinia IgG will also be measured at time points up to 2 weeks post vaccination.

As will be obvious to those skilled in the art, the above described study may also be used to test the efficacy of vaccinia virus antibodies administered via alternate routes, such as intramuscular, intranasal, and other examples described above. The above study may also be used to determine the effectiveness of other formulations of Orthopoxvirus antibodies such as OPVIG and OPVIF. Such a study may involve challenging the subjects in an Orthopoxvirus vaccine other than vaccinia virus. For example, if an OPVIG is produced using an attenuated variola virus vaccine, the above study may be performed by challenging the volunteers with the variola vaccine and the OPVIG.

Preclinical Studies

Animal Model Rationale

Humans cannot be used to test the efficacy of drugs for protection against virulent Orthopoxvirus. There is no ethical way to intentionally challenge humans with a virulent Orthopoxvirus and there are no naturally occurring reservoirs of human infection. Thus, an appropriate animal model needs to be established. A number of studies have made use of adult murine models of infection in which the virus is administered by different routes [Boyle J. J., et al, Antimicribiol Agents and Chemotherapy 1966 pp 536-539, Bray et al, J Infect Dis 2000, 181:10-19; De Clercq and Somer, Applied Micro 1968;16:1314-1319; De Clercq et al., Proc Soc Exp Biol Med 1976; 151:487 90; Williamson et al., J Gen Virol 1990;71:2761-2767). Four species of Orthopox virus: variola, monkeypox, cowpox, and vaccinia viruses, can cause infections in humans (Fenner, F. Poxviruses. In Fields Virology (B. N. Fields, D. M. Knipe, P. M. Howley, eds.). 1996, 2673-2699.). However, variola and monkeypox viruses do not cause diseases in adult mice and surrogate viruses, such as vaccinia or cowpox, are required to simulate Smallpox infection.

Vaccinia Tail Infectivity Model:

Inoculation of vaccinia virus into the tail vein of mice was shown by Boyle [1966] to produce a self-limiting infection that results in discrete dermal lesions along the entire surface of the tail. Enumeration of the lesions is facilitated by staining with a combination of fluorescein and methylene blue that stains necrotic tissue [Boyle J. J., et al, Antimicribiol Agents and Chemotherapy 1966 pp 536-539]. This method has been shown to be highly sensitive and reliable for assessing the efficacy of antiviral compounds [Boyle J. J., et al, Antimicribiol Agents and Chemotherapy 1966 pp 536-539; De Clercq and Somer, Applied Micro 1968;16:1314-1319]. Furthermore, the tail infectivity model represents a simpler and more objective way of assessing the protective efficacy of antiviral compounds than experiments based on mortality rate.

This model of infectivity closely resembles the human vaccination process in that dermal lesions occur on the tail of the mouse similar to the vaccination site lesion that occurs in humans. Furthermore, in both mouse and human, administration of vaccinia via these routes typically causes self-limiting infections.

The viral challenge dose that elicits a minimum of 50 pocks per tail was identified. This dose facilitates easy and accurate enumeration of the lesions on the murine tail and was used in subsequent studies to evaluate the antiviral activity of VIG. A range of doses of VIG from 3,000 U to 30,000 U/Kg body weight were compared for their ability to reduce pock formation using this model. The results demonstrated that administration of VIG resulted in a statistically significant decrease in the numbers of pox that formed on the mouse tail.

VIG Used in Combination with Antiviral Agents

Studies in animal and cell culture models of Orthopoxvirus infections have proven the effectiveness of antivirals in treating infections from various species of viruses from the Orthopoxvirus family including camelpox, cowpox, monkeypox, and vaccinia viruses. Specific antivirals that have been shown to be effective include Cidofovir™ (Smee D F, et al, Antiviral Res (2000) 47:31 71-7; Smee D F, et al, Antivir Chem Chemother (2001) 12:171-6) and other acyclic nucleoside phosphonate derivatives (Snoeck et al, Antimicrob Agents Chemother (2002) 46:11 3356-3361), lamivudine (3TC), famciclovir, penciclovir, ribavirin and mycophenolic acid (Smee D F, et al, Antivir Chem Chemother (2001) 12:6 327-35; Smee D F, et al Antivir Chem Chemother (2000) 11:4 303-9). However, such antiviral agents are often associated with severe side effects that may prove deadly in patients severely ill with an Orthopoxvirus infection. Additionally, antivirals require several days of treatment before a therapeutic effect is realized. In patients that present with an advanced Orthopoxvirus infection, such a delay may prove lethal. Finally, Orthopoxvirus strains have also been described that are resistant to such antiviral agents (Smee D F, et at, Antimicrob Agents Chemother (2002) 46:5 1329-35).

Mice in the above described vaccinia tail infectivity model were treated either with Cidofovir™ alone, VIG alone, or a combination of both Cidofovir™ and VIG. Surprisingly, the combination of Cidofovir™ and VIG resulted in a statistically significant lower number of pox on the tails of the mice compared to either therapy alone. Such a combination therapy would prove efficacious in treating patients with life threatening Orthopoxvirus infections than either therapy alone. Such a combination therapy could also allow a lower dose of the antiviral agent, which could reduce the side-effects from the antiviral agent. Additionally, should an antiviral resistant strain of Orthopoxvirus emerge, it may not be possible to determine which patients are infected with the resistant strains and which are infected with the sensitive strains. Another embodiment of the present invention is the treatment of an Orthopoxvirus infection with a combination of an antiviral agent such as Cidofovir™ and IV-VIG.

A combination therapy may be important in that the patient infected with an Orthopoxvirus would receive the benefit of the therapy with VIG. As will be obvious to those skilled in the art, any antiviral agent that has shown efficacy in treating an Orthopoxvirus infection could be used in combination with a VIG, OPVIG or OPVIF.

Kit Containing VIG and Antiviral

A pharmaceutical combination in kit form may be provided which includes in packaged combination a carrier means adapted to receive a container means in close confinement therewith and a first container means including a pharmaceutical immune globulin formulation effective against an Orthopoxvirus and an antiviral agent effective against an Orthopoxvirus. In such a kit, the immune globulin formulation and antiviral agent compositions may be in different administrable forms. For example, the antiviral agent may be in an orally administrable form such as tablet, pill, capsule or powder form, while the immune globulin formulation may be in a form suitable for administration by injection, i.e., in solution form. Such kits can further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, can also be included in the kit.

The present invention also includes use of combination compositions as presented herein further in combination with other medical compositions intended for the treatment of those with Orthopoxvirus infections set forth herein.

In one embodiment, the pharmaceutical combination in kit form is directed to an adverse reaction to Smallpox vaccination or an infection with the Smallpox (variola) virus and includes VIG and Cidofovir™.

Use of VIG Prophylactically

Vaccinia vaccine should not be administered if certain conditions are present in the individual or in their household contacts. Such conditions include:
a history or presence of eczema or other skin conditions because of the increased risk for eczema vaccinatum;
persons with other acute, chronic, or exfoliative skin conditions (e.g., atopic dermatitis, burns, impetigo, or varicella zoster);
pregnancy due to the risk of fetal vaccinia infection which usually results in stillbirth or death of the infant soon after delivery;
persons with immunodeficiency diseases and/or immunosuppression (e.g., as occurs with leukemia, lymphoma, generalized malignancy, solid organ transplantation, cellular or humoral immunity disorders, or therapy with alkylating agents, antimetabolites, radiation, or high-dose corticosteroid therapy [i.e., ≧2 mg/kg body weight or 20 mg/day of prednisone for ≧2 weeks]), hematopoietic stem cell transplant recipients who are 24 months post-transplant, and hematopoietic stem cell transplant recipients who are >24_months post-transplant but who have graft-versus-host disease or disease relapse;
persons infected with HIV;
infants and children;
persons with allergies to vaccine components, the currently available vaccinia vaccine (i.e., Dryvax®) contains trace amounts of polymyxin B sulfate, streptomycin sulfate, chlortetracycline hydrochloride, and neomycin sulfate which may result in anaphylactic reactions; and
persons who anticipate being exposed to an infectious Orthopoxvirus within a timeframe insufficient to develop immunity by vaccination.
   In the event of a release or outbreak of an Orthopoxvirus, a proposed treatment modality is the administration of VIG prophylactically into such individuals to prevent infection. There is some evidence that VIG-IM has proven effective in the prophylaxis or Feery B J, Vox Sang (1976) 31:68-76; Barandun S, et al, Monogr Allergy (1975) 9:39-60. An experimental model may be used to demonstrate the effectiveness of a VIG-IV formulation in preventing an Orthopoxvirus infection. Recently, an intranasal model of infection has been developed in adult mice (Bray, J Infect Dis 2000, 181:10-19). This model has the advantage of simulating the route of infection that would take place in a natural Orthopoxvirus infection or that would occur if Smallpox were used as a biological weapon. The endpoint of this infection model is death, which is similar to variola major infections in humans with case fatality rates of 20% or greater (Henderson and Moss, Smallpox and Vaccinia. In Vaccines (S. A. Plotkin and W. A. Orenstein, eds), 1999 p. 74-97).

The strain that is used in this infection model is Cowpox strain Brighton. Although both vaccinia and cowpox are able to cause disease in adult mice, it has been previously demonstrated that the Western Reserve strain of vaccinia virus causes lethal encephalitis in mice after intranasal exposure [Williamson et al, J Gen Virol 1990;71:2761-2767]. This disease manifestation is markedly different from that which is observed in humans with severe Orthopoxvirus infections where the central nervous system is not affected. Conversely, the Cowpox strain Brighton is lethal for mice and causes a fatal pneumonitis upon intranasal administration. But similar to the human infection, it does not affect the central nervous system of the mouse [Thompson et al., Virology 1993-197:328-338]. Bray and coworkers [J Infect Dis 2000, 181:10-19] revealed that intranasal infection of mice with Cowpox strain Brighton elicited pock formation throughout the lungs causing bronchiolar mucosal edema, small-airway obstruction, and hemorrhage.

The viral challenge dose that results in 100% lethality by day 21 will be identified and used in subsequent treatment studies. A range of doses of VIG will be tested to identify the minimum dose resulting in 100% protection. Subsequent experiments will investigate the impact that pre- and postexposure treatment and route of administration (IV versus IM) have on the experimental outcome.

As will be obvious to those skilled in the art, similar studies may be performed to test the effectiveness of OPVIG and OPVIF.

Recombinant Vaccinia Viruses

Vaccinia virus can be genetically engineered to contain and express foreign DNA with or without impairing the ability of the virus to replicate. Such foreign DNA can encode protein antigens that induce protection against one or more infectious agents. Recombinant vaccinia viruses have been engineered to express immunizing antigens of herpesvirus, hepatitis B, rabies, influenza, cancer antigens, human immunodeficiency virus (HIV), and other viruses (Kieny M P, et al, Nature 1984;312:163-6; Smith G I L, et al Nature 1983;302:490-5; Smith G L, et al, Proc Natl Acad. Sci USA 1983;80:7155-9, Zagury D, et al, Nature 1987;326:249-50).

Since many of these clinical trials are performed on patients who have undergone previous treatment regimens that may have left the patient immunosuppressed, the potential exists that an attenuated recombinant vaccinia may cause: a life threatening infection in the patient. In the event of such a complication, such patients may be treated with immune globulin formulations against such vaccinia virus or Orthopoxvirus such as VIG, OPVIG or OPVIF that may control or treat the infection. The possibility also exists that a “breakout” recombinant vaccinia virus may be generated in the course of the gene therapy studies; VIG, OPVIG or OPVIF may be used alone or in conjunction with vaccinia or Orthopoxvirus vaccination to treat the affected patient and any infected or exposed medical personnel.

During human trials of recombinant vaccines, physicians, nurses, and other health-care personnel who provide clinical care to recipients of these vaccines could be exposed to both vaccinia and recombinant viruses. This exposure could occur from contact with dressings contaminated with the virus or through exposure to the vaccine. The risk for transmission of recombinant vaccinia viruses to exposed health-care workers is unknown. However, the potential does exist of non-highly attenuated vaccinia viruses or recombinant viruses derived from these strains being transmitted to health-care personnel. Such health care workers may be treated with VIG, OPVIG or OPVIF prophylactically to prevent infection with such viruses.

Recombinant Orthopoxvirus may also be generated as bioterrorist weapons. To guard against such a scenario, researchers may generate recombinant virus from any member of the Orthopoxvirus genus and may be engineered to increase its virulence or increase the infected cells' resistance to antiviral drugs, or alter the host immune system. One example of such experiments is engineering the ectromelia virus to express IL-4, which suppresses primary antiviral cell-mediated immune responses and inhibits the expression of immune memory response (Jackson, R. J., et al, Journal of Virology (2001), Vol. 75(3) p. 1205-1210). Researchers working on such viruses, or exposed to such viruses, may be treated with VIG, OPVIG or QPVIF either therapeutically of prophylactically. Infections with such a recombinant Orthopoxvirus may be treated with VIG, OPVIG or OPVIF in conjunction with vaccinia vaccination.

Having generally described the invention, the same will be more readily understood through reference to the following examples which are provided by way of illustration, and it are not intended to be limiting of the present invention.

### EXAMPLES

### Example 1

Colorimetric Assay For Neutralizing Antibodies

The BS-C-1 cell line, available from ATCC (ATCC Catalogue No. CCL-26), originating from African green monkey kidney, are grown in Dulbecco's Modified Eagle's Medium (DMEM) or RPMI-1640 medium without phenol red, supplemented with 5% fetal calf serum (FCS). Cells are seeded at 4.5×10⁴ cells per 180 μl in each well of a 96-well plate. Confluent cell monolayers are formed within 3 to 4 days at 37° C.

The Lister strain of vaccinia virus, available from the ATCC (ATCC Catalogue No. VR-862) or the NYCBOH-Wyeth vaccinia strain (ATCC Catalogue No. VR-325), is applied in the neutralization assays. Two-fold dilutions of sera from donors vaccinated with the vaccinia virus are performed in polyvinyl-chloride transfer plates and incubated with 100 Plaque Forming Units (PFU) of vaccinia virus for 1 hr at 37° C. Each mixture is then directly transferred to a well in the 96 well plate containing preformed monolayers of BS-C-1 cells. Two controls are also included: control infected cells wherein 100 PFU of vaccinia virus that has not been exposed to neutralizing antibodies is added to the BS-C-1 cells, and control uninfected cells wherein no vaccinia virus is added to the BS-C-1 cells. After incubation for 3 to 4 days at 37° C., or until more than 80% of the control infected cells are rounded, the medium is replaced with 90 μl of phenol red deficient RPMI 1680 medium and 10 μl WST-1 (Roche Molecular Biochemicals) and incubated for 2 hrs at 37° C. The 96 well plate is then shaken thoroughly for 1 minute on a shaker, and the 96 well plate is read at 440 nm using an automatic spectrophotometer (Microwell System, Organon Teknika, Austria). The optical-density reading that relates to protection of 50% of the BS-C-1 cells is calculated as follows: (50%×[optical-density reading of uninfected cells minus optical-density reading of the control infected cells]). The 50% activity of a serum is determined by its dilution required for protection of 50% of the BS-C-1 cells (as calculated above).

Plaque Reduction Neutralization Test Test Assay (PRNT)

PRNT assays are among the most sensitive methods for detecting virus-specific neutralizing antibodies. The procedure involves incubating serial dilutions of a sample containing antibodies to vaccinia virus with a constant amount of virus, then infecting monolayers with the preincubated antibody-virus mixture. The number of plaques that result in the monolayers are enumerated and then compared to the number of plaques that form in a monolayer infected with virus alone in order to determine the neutralizing titre. A reduction in the number of plaques by the test sample as compared with the virus alone indicates neutralization or antiviral activity. The measure of neutralization is expressed by a plaque reduction neutralization titre (ND₅₀) which is the reciprocal of the sample dilution that reduces the plaque count by 50%.

A plaque assay was developed to determine the titre of the vaccinia virus strain IHD stock. For optimum results in a plaque assay, it is important that a confluent monolayer be inoculated with virus to provide the best results, and in order that gaps in the monolayer are not inadvertently counted as plaques. The volume used in the inoculation should be at a minimum to allow for contact of the virus with the cell surface, but not too low where drying of the monolayer may occur during inoculation.

A semisolid overlay is used after inoculation of a plaque assay in order to localize the virus infection in the monolayer. Agar overlays are often used for this purpose. Agar overlays ranging from 1.0-1.5% can be used. One alternative is to use 1% Sephadex® (Pharmacia) suspension in culture media. The timing to allow plaque development must be such that the plaques can be easily counted, but not too large such that there is overlap and individual plaques cannot be clearly distinguished. Preferably, the overlay and staining procedure is such that the monolayers remain intact and plaques will be readily viewed. When the plaques have formed, the monolayers were stained to visualize the plaques. A number of staining methods can be used including neutral red (Molecular Probes, Inc. Eugene, Oreg. USA), XTT (2,3-bis-(2-methoxy-4-nitro-5-sulfophenyl)-2H-tetrazolium-5-carboxanilide) (Molecular Probes, Inc. Eugene, Oreg. USA), MTT (3-(4,5-dimethylthiazol-2-yl)-2,5-diphenyltetrazolium bromide) (Molecular Probes, Inc. Eugene, Oreg. USA), WST-1 and crystal violet. These dyes may be added directly to the overlay, or as an additional agar overlay. Following staining, the agar was removed and the monolayers fixed and stained.

BS-C-1 cells (American Type Culture Collection [ATCC] CCL-26) were plated at a cell seeding density of 7.5×10⁵ cells/well in 6-well cell culture plates. It was previously determined that this seeding density produced confluent monolayers of BS-C-1 cells one day after seeding. The monolayers were subsequently inoculated with virus and they were observed for plaque formation 2 to 5 days following inoculation. It was determined that after two days, the size of the plaques formed were large enough to allow for ease of enumeration, while providing a reliable count. Based on the virus reference titre, aliquots of virus were diluted in culture medium to achieve approximately 100 plaque forming units/well.

A two-fold dilution series of the reference control VIG standard was prepared. Equal volumes of vaccinia virus inoculum and each reference control dilution were mixed and preincubated at 37±2° C./5% CO₂ for approximately 60 minutes, to allow antibodies, if present, to bind to the virus. Each of the preincubation mixtures was inoculated in triplicate onto confluent monolayers of BSC-1 cells. Negative controls consisting of media alone and positive controls consisting of virus alone were run in parallel.

The inoculated cultures were incubated at 37±2° C./5% CO₂ for approximately 60 minutes to allow the virus, if present and not neutralized, to infect the BS-C-1 cells. Subsequently, a 1% Sephadex® and media solution was overlaid onto the monolayers. The cultures were maintained for a further 2 days at 37±2° C./5% CO₂, at which time the staining of the monolayers was performed.

After staining the monolayers, plaques in each well were enumerated. Plaques were observed as clear areas against a background of stained cells.

For each individual assay, the mean of the triplicate values for each dilution was calculated and recorded. The dilution of the reference control standard that resulted in a 50% reduction in the total number of plaques produced in the positive control was determined. The reciprocal of this dilution was reported as the neutralization titre (ND₅₀) of the reference control standard.

To determine the relative potency using the PRNT assay, parallel PRNT assays were set up with the above runs of the assay using a pre-diluted reference control standard (sample). The pre-diluted sample is prepared then serially diluted and used in the PRNT assay. An ND₅₀ is calculated for the pre-diluted sample and compared to that achieved with the reference control standard to determine relative potency. The difference in the resulting titres for each should be related to the initial predilution of the reference control standard.

ELISA method for detection of Vaccinia virus binding antibodies.

An ELISA assay may be used to detect antibodies that bind to the vaccinia virus lysate bound to a microtitre plate. To prepare the vaccinia virus lysate, BS-C-1 cells were grown at 37° C. to about 80% confluence, and infected with vaccinia virus strain WR (ATCC Catalogue No. 1354) at a multiplicity of infection (MOI) of 0.1 for one hr at 37° C. The infected BS-C-1 cells were then incubated for an additional 48 hrs at 37° C. to allow the vaccinia virus to propagate. The infected BS-C-1 cells were harvested and pelleted by centrifugation at 3000 rpm for 5 min. The cell pellet was resuspended in 100 mM of Tris-HCl, pH 8.0, and an equal volume of cell lysis buffer was added to lyse the cells [cell lysis buffer: 100 mM Tris-HCl, pH 8.0; 100 mM NaCl; 0.5% Triton X-100; 0.2 mM phenylmethylsulfonyl fluoride (PMSF, added just prior to use)]. The lysate was buffer-exchanged with 1×PBS (10 mM Na₂P0₄, 150 mM NaCl, 0.01% NaN₃, pH 7.4) using a 10 kDa centrifugal device until the concentration of Triton-X-100 was reduced to <0.002% and PMSF was reduced to <0.001 mM. An approximate protein concentration was determined by BCA protein assay (bicinchoninic assay, Pierce Chemical Rockford, Ill., USA). The vaccinia virus lysate was stored at −80° C.

The above described vaccinia lysate was diluted to 0.05 ng protein/mL in PBS buffer (pH 7.2 to 7.6). The wells of a 96-well microtiter plate were coated with the vaccinia lysate by adding 125 μL of lysate/well and incubating overnight at 2-8° C. The lysate solution was decanted, and the wells were washed three times with a washing solution (PBS with 0.1% Tween 20). 350 μL of Super Block (Scytek Laboratories Inc., Logan, Utah, USA) is added to each well and the plate is incubate for 3 hrs at room temperature. The Super Block™ is decanted and the wells are washed three times with the above washing solution. 150 μL of StabilCoat™ (SurModics Inc., Eden Prairie, Minn., USA) is added and the plates are incubated for 30 min at room temperature. The plates were allowed to dry and may either be used immediately or sealed and stored at 5° C.

The ELISA assay was performed by adding 100 μL of standards, controls, and sample dilutions to designated wells. Dilutions were made in an assay dilution buffer solution [400 ml 1×D-PBS, 100 ml Super Block, 10 g Non fat milk powder, 500 μL Tween 20, 250 μL Proclin 300, 4 ml normal human plasma (Heparinized and Citrated, commercially available) negative for vaccinia antibody]. To remove the interference effects observed with plasma, the minimum plasma sample dilution is 1/100. Standards and positive controls were prepared by initial dilution of positive plasma samples 1/100 using plasma-free dilution buffer, followed by further dilution with 1% plasma dilution buffer. Standards were prepared by dilution of Baxter VIG standard (Lot No. 0448A101AE), assigned an arbitrary value of 10,000 U/mL, to a range covering 0.01 to 4 U/mL. Test samples were prepared by initial dilution to 1/100 with plasma-free dilution buffer followed by further dilution with 1% plasma dilution buffer to the appropriate range, which may be from 1/100 to >1/10000. Negative control was prepared by dilution of normal negative plasma to 1/100 with plasma-free dilution buffer. Once the antibody source is added to the antigen coated wells of the microtitre plate, the plate is incubated for 1-2 hrs at 37° C.

The wells are then decanted and washed with the washing solution, as described above. To all wells 100 μL of Peroxidase-conjugated AffiniPure Goat Anti-Human IgG, Fcγ Fragment Specific (Jackson Immuno Research Laboratories, West Grove, Pa., USA) at a dilution of 1/5000 in assay dilution buffer is added and incubated at room temperature for 1 hr. All wells are then decanted again and washed with the washing solution. To all wells 125 μL of substrate solution [100 mM Sodium Acetate Trihydrate (pH 5.5), 0.0045% w/v hydrogen peroxide, 0.01% (w/v) 3,3′,5,5′-tetramethylbenzidine (Sigma-Aldrich, USA)] is then added. The substrate solution in the wells is then incubated for 15 minutes at room temperature protected from light. The reaction is then stopped by adding 100 μL of 2M sulfuric acid solution and the plate read with a 96-well plate reader/spectrophotometer at 450 nm and at 650 nm wavelength. A standard curve was plotted as linear optical density response (difference between the readings at 450 nm and at 650 nm wavelength) vs log-dose concentration. The standard curve can then be fitted with an iterative 4-parameter logistic analysis. Controls and samples are then interpolated using the logistic equation derived from the standard curve. The sample concentrations can then be corrected by dilution rate.

ELISA and PRNT Results Correlated

To make a VIG product, the plasma from donors and the final VIG product are assayed to determine the amounts of vaccinia virus specific antibodies. The number of antibodies specific to vaccinia virus can be determined by an ELISA assay or by the PRNT assay. The ELISA assay measures the amount of antibodies that bind to the vaccinia antigen while the PRNT assay is a functional assay that measures the amount of antibodies that actually neutralize the virus. It was previously assumed that the results of a PRNT assay were more accurate (Lublin-Tennenbaum et al., 1990, Viral Immunology 3: 19-25).

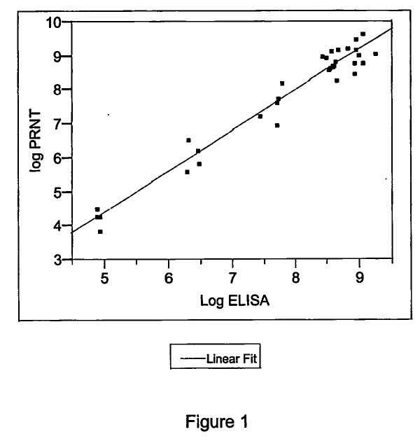

Samples of a VIG were diluted to a specific concentration. Parallel samples were assayed by ELISA and PRNT, and compared to a standard VIG that was assigned a titre of 10,000 U/ml. A Bivariate Fit of log By Log graph (FIG. 1) demonstrates the correlation between the results of the ELISA assay and the PRNT assay. Correlation coefficient of 0.98 indicates a very high level of correlation. Thus, the plasma and VIG may be screened by either ELISA or PRNT assay for the presence of vaccinia antibodies.

### Linear Fit log PRNT=−1.597741+1.2015617 Log ELISA

Requirement to Vaccinate Plasma Donors

The above-described ELISA assay was used to test plasma samples from 5 naïve donors (i.e. donors that have never been vaccinated for Smallpox). All the plasma samples tested from these naïve donors exhibited vaccinia-binding activities near the base line (less than 3 U/mL). In comparison, a plasma pool from donors vaccinated against Smallpox during childhood, but not subsequently revaccinated, had an activity of approximately 80 U/mL. These data suggest that a vaccinia immune globulin (VIG) must be manufactured from the plasma of donors that have been previously exposed to an Orthopoxvirus infection or an Orthopoxvirus vaccine.

The plasma pool having an activity of approximately 80 U/mL was used to manufacture a Vaccinia Immune Globulin (VIG) solution (by the method described herein). The finished VIG product resulted in a 4.5% protein solution with an activity of around 800 U/mL, or approximately 8% of the activity of the VIG standard solution produced by Baxter. A VIG with 800 U/ml activity may be suitable for certain applications, such as for intravenous administration.

The above results suggest that to produce a 5 g % (w/v) protein VIG comparable in activity to the VIG standard solution produced by Baxter (i.e. 10,000 IU/ml), donor plasma having an activity of about 1,000 IU/ml should be collected. The above calculations are predicated on the assumption that the ELISA assay will correlate with the PRNT assay. As stated previously, a more informative assay is an assay that detects the levels of protective antibodies, such as the PRNT assay. In a preferred embodiment, a VIG should be produced wherein the levels of protective antibodies are comparable to the levels found in the standard solution produced by Baxter. However, the above results suggest that revaccination of the plasma donors with the Smallpox vaccine might be required to elevate the levels of vaccinia virus antibodies, and thus produce a VIG with increased potency.

Immunization of Donors

Donors were adults in generally good health, who had previously been immunized with vaccinia vaccine, as evidenced by a visible vaccination scare and either a written or oral history of being vaccinated. Dryvax®, the vaccinia (Smallpox) vaccine, available through the US Centers for Disease Control and Prevention in Atlanta, Ga., is a lyophilized, live-virus preparation of infectious vaccinia virus (Wyeth Laboratories, Inc., Marietta, Pa.). Vaccinia vaccine does not contain Smallpox (variola) virus. The vaccine had been prepared from calf lymph with a seed virus derived from the New York City Board of Health (NYCBOH) strain of vaccinia virus and has a minimum concentration of 10⁸ pock-forming units (PFU)/ml. Vaccine was administered by using the multiple-puncture technique with a bifurcated needle, supplied with the vaccine.

Typically, the site of vaccination is on the shoulder. No preparation of the skin at the site of injection is required. However, if site is obviously dirty, a cloth moistened with water may be used to wipe the site. Use of a disinfectant can kill the vaccine virus. Withdrawal of vaccine from ampoule. A sterile bifurcated needle (which is supplied with the vaccine and must be cool) is inserted into the ampoule of reconstituted vaccine. On withdrawal, a droplet of vaccine, sufficient for vaccination, is contained within the fork of the needle.

Application of Vaccine to the Skin

The needle is held at a 90 degree angle (perpendicular) to the skin. The needle then touches the skin to release the droplet of vaccine. For both primary and revaccination, 15 up and down (perpendicular) strokes of the needle are rapidly made in the area of about 5 mm in diameter (through the drop of vaccine deposited on the skin). The strokes should be sufficiently vigorous so that a trace of blood appears at the vaccination site. If a trace of blood does not appear, the strokes have not been sufficiently vigorous and the procedure should be repeated. Although it is desirable not to induce frank bleeding, the proportion of successful takes is not reduced if bleeding does occur. No dressing should be used after vaccination.

Safety of Revaccinating Plasma Donors with Dryvax® Vaccine

Since systematic vaccination for Smallpox ended in North America nearly 30 years ago, for the vast majority of the population, primary Smallpox vaccination occurred more than 30 years ago. To determine the safety of revaccinating with Smallpox vaccine 30 years after primary vaccination, 20 human study subjects with a previous history of Smallpox vaccination, were revaccinated with Dryvax® according to the procedure described above. The study subjects were then monitored for 8 weeks, and their observed reactions to the vaccine are summarized in Table 1. The typical “pox reaction” following vaccination with Dryvax® evolved with the following characteristics.
1. Erythema surrounding the vaccination site can occur as early as 30 minutes following vaccination. In these 20 subjects erythema was seen in 100% of the cases, the mean duration of the erythema was 14 days and had a peak incidence of occurrence at day 7.
2. Swelling around the vaccination site was observed in 95% of subjects, was most frequently reported on day 7 and lasted a mean of 10 days.
3. Blistering (vesiculation) occurred in only 65% of the vaccinated subjects, lasted a mean of 5.5 days and occurred on day 2 most frequently.
4. Pustulation of the vesicle occurred in 80% of subjects, peaked 9 days following vaccination and lasted a mean of 5 days. It is interesting to note that in 5 subjects, pustulation was reported without vesiculation.
5. Crusting of the lesion occurred in 95% of subjects, was seen most frequently at day 14 and lasted a mean of 7 days.
6. Pruritis of the vaccination site was reported 90% of the time, was seen most frequently at day 7 and had a mean duration of 8.5 days.

During the study period, no unexpected adverse events were observed in these 20 subjects, and there were no serious vaccine complications that are reported in the literature such as accidental inoculation to other sites, eczema vaccinatum, vaccinia necrosum or vaccinia encephalitis. These results indicate the safety of revaccinating with Smallpox vaccine to stimulate antibody production in plasma donors.

Efficacy of Revaccinating Plasma Donors on Vaccinia Virus Antibody Levels

Following vaccination of the 20 human subjects described above, plasma was collected from the subjects by automated plasmaphoresis devices PCS®2 System by Haemonetics Corporation (Braintree, Mass.) and Autopheresis-C from Baxter (Round Lake, Ill.). The average unit collected is 0.8 liters. The plasma was screened for the presence of disease indications as required by the FDA, and the levels of vaccinia binding antibodies were determined by the ELISA assay described above. Table 2 summarizes the results. The results indicate that there was a significant increase in the levels of vaccinia-binding antibodies at 10 days post vaccination, and the elevated antibody levels persisted until approximately 60 days post vaccination. The vaccinia-binding antibody levels significantly decrease approximately 7 weeks post vaccination, though they remain elevated compared to pre-vaccination levels. This would suggest the optimal time to collect plasma from the donors for the preparation of VIG is during the period when the vaccinia binding antibody levels are high, which in this application is between 10 and 60 days post revaccination.

All 20 of the subjects demonstrated a typical “pox reaction” following vaccination (i.e. the development of a pustular lesion at 6-10 days post vaccination). However, approximately 20% of the plasma donors demonstrated a very low antibody response to vaccination (less than 100 U/ml) as determined by the ELISA assay. This suggests that the current practice of observing the development of a pustular lesion at 6-10 days post vaccination is not an accurate indication of the “take” of a vaccine. Therefore, the plasma samples may be screened for vaccinia-binding antibody levels or for protective antibodies, prior to use of the plasma for the production of VIG. Such results also suggest that the effectiveness of vaccination should be determined using an assay to detect the levels of vaccinia binding antibodies or protective antibodies.

Time Course For Vaccinia Antibodies in Donor Plasma

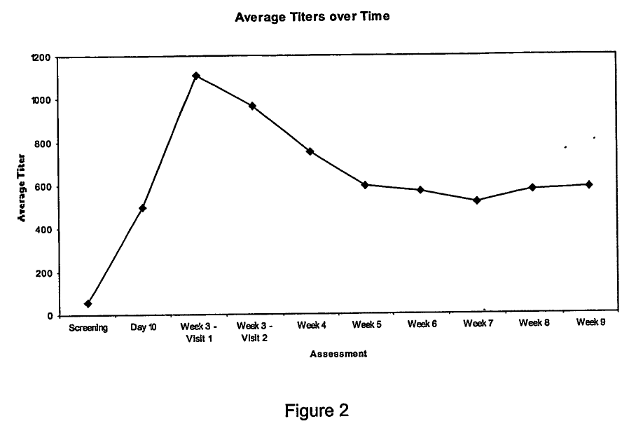

Plasma donors were selected if they had medical documentation of a previous Smallpox vaccination or a Smallpox vaccination scar. The plasma donors were vaccinated and plasma was drawn twice weekly for nine weeks. The plasma was assayed for vaccinia antibodies by an ELISA assay, as shown in FIG. 2.

Re-Vaccinating the Plasma Donors

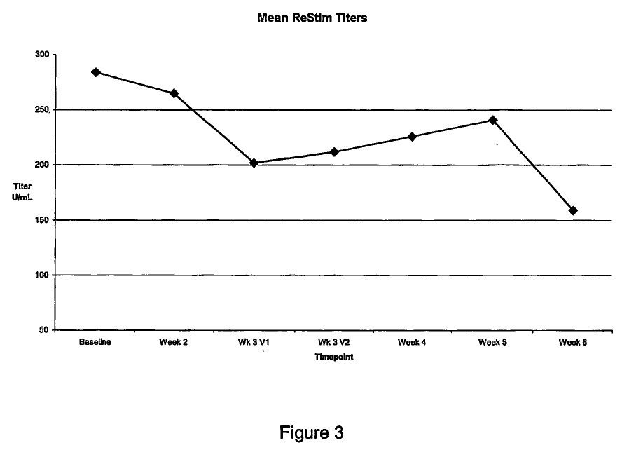

Since the titres of vaccinia antibodies in the donor plasma, an attempt was made to revaccinate the plasma donors with the Smallpox vaccine. Plasma donors were revaccinated with the Smallpox vaccine at least 12 weeks after the original vaccination. Plasma samples were collected and assayed for vaccinia antibodies. The results are shown in FIG. 3. There was no increase in the titre of vaccinia antibodies.

Plasma from donors is periodically tested for the titre of vaccinia antibodies by ELISA by the method described above. In one embodiment, plasma units with vaccinia antibody levels below 200 U per ml is not used. Plasma units are pooled, and the pools tested for vaccinia DNA by PCR and may be tested for vaccinia antibody levels.

Manufacture of VIG

Plasma Pool Preparation

The frozen plasma units are allowed to thaw at room temperature, and the plasma is transferred to the pooling tank. The pooled plasma is diluted in the tank with water suitable for injection into human patients (WFI) (2 parts plasma: 1 part WFI) and mixed. Dextran sulphate, dissolved in WFI, is added to the pooled plasma tank to a final concentration of 0.03% (w/w) and mixed. The plasma is then filtered through cellulose filter pads (Seitz 700 or equivalent).

Anion-Exchange Chromatography

The anion exchange chromatography column is prepared in a cold room (approximately 8° C.). DEAE-Sephadex™ A50 resin (Pharmacia) is allowed to swell in 0.025 M potassium phosphate buffer pH 7.5 for 2-24 hours in a chromatography column. The DEAE-Sephadex™ is then treated sequentially with 1 M NaOH and 0.08 M H₃PO₄. The column is equilibrated by flushing with phosphate buffer.

The Dextran-Sulphate treated plasma is applied to the DEAE-Sephadex column. When all the plasma has been applied, the column is eluted with equilibrating buffer (0.025M phosphate, pH 7.5) at the same flow rate. The column effluent absorption at UV₂₈₀ is monitored and the UV₂₈₀ absorbing peak (IgG effluent) is collected. The collected IgG effluent is filtered through two 35 nm Planova filters in series, and collected.

Ultrafiltration

The IgG effluent collected from the DEAE-Sephadex column is concentrated to approximately 0.5× the original volume by ultrafiltration, using a Millipore Pellicon ultrafiltration system (or equivalent) with 10,000 MW cut-off polysulfone cassettes.

Addition of Solvent/Detergent Solution

Lipid enveloped viruses are inactivated by a solvent detergent method. tri-n-butyl phosphate (TNBP) and Triton X-100 are added to a final concentration of 0.3% TNBP: 1.0% Triton X-100 and the solution is mixed for 4-4.5 hours at 4° C. The TNBP and Triton X-100 is removed by reverse phase chromatography using a column packed with C-18 resin and eluted with WFI. The column effluent absorption at UV₂₈₀ is monitored and the UV280 absorbing peak, containing the IgG, is collected.

0.2 Micron Filtration and Dispensing

The IgG is pH adjusted to 5.5 with 1 M H₃PO₄, diafiltered with >4 volumes of cooled WFI using a Millipore Pellicon ultrafiltration system (or equivalent) with 10,000 MW cut-off polysulfone cassettes. Maltose is added to the IgG from the diafiltration step to a final concentration of 10% w/w, and polysorbate 80 is added to a final concentration of 0.03% w/w and mixed. The formulated IgG solution is aseptically filtered through a 0.2 micron filter, stored at 2-8° C. until QC testing is complete and aseptically dispensed into vials. The vials are stoppered, sealed with aluminum caps and stored at 2-8° C. until ready for packaging. The immune globulin formulation thus produced (VIG-CAN-1, will be used for further studies as outlined below)

QPCR Analysis of Plasma and VIG Product

The target DNA sequence is a region of the B18R gene encoding the interferon-alpha/beta receptor from the vaccinia virus strain Wyeth (GenBank Accesssion # AJ269556). A set of forward and reverse primers were designed using Primer Express software version 2.0 from Applied Biosystems. The sequence of the forward primer is: 5′-TTCGGAGGCACAATGAATGA-3′ (SEQ ID NO. 1), and anneals between residues 240 and 259. The sequence of the reverse primer is: 5′-TGTCTTCAATAGGAGGACACTTTGC-3′ (SEQ ID NO. 2), and anneals between residues 312 and 288.

A vaccinia DNA standard is a pUC119 plasmid encoding a 113 bp insert from residues 220 to 332 of the GenBank plasmid. A cloned internal control standard differs from the vaccinia DNA standard by a four base pair deletion (GAGC) from residues 276 to 279 and a four base pair insertion (AGCG) between residues 284 to 285.

The sequence of the vaccinia MGB probe is: 5′-6-carboxyfluorescein (FAM)-CTCTAGGAGAGCCATTC-MGB-3′ (SEQ ID NO. 3), anneals between residues 268 and 284 of the vaccinia DNA standard. The sequence of the internal control MGB probe is: 5′-VIC-CTCTAGGACATTCAGCG-MGB-3′ (SEQ ID NO. 4). In the presence of the target and internal control sequences, two amplicons are produced that are 73 bp and 69 bp, respectively.

The QPCR is analyzed on an ABI PRISM® 7700 Sequence Detection System (Applied Biosystems, Foster City, Calif.). Distinction is made between the Vaccinia target sequence and the internal control sequence due to a four base pair mismatch in the DNA sequence of the two Taqman MGB probes as well as the use of two different reporter dyes to label the target and the internal control, namely, the FAM signal for vaccinia target sequence detection and the VIC signal for internal control sequence detection. Simultaneous amplification with these two Taqman MGB probes results in a multiplex QPCR assay that provides simultaneous detection of the vaccinia target sequence and the internal control sequence in the same closed tube. The main advantage of using an internal control sequence designed with a different probe hybridization site is that it makes the QPCR assay suitable for quality control applications where co-detection of the target and the internal control is required in the prevention of false negative.

A standard curve was prepared in a related sample matrix (i.e. plasma) extracted by QIAamp® UltraSens™ Virus Kit (Qiagen) and is included in every QPCR run. It consists of five concentrations of the DNA standard spanning a range above and below the expected copy number in the test samples: 10⁶, 10⁵, 10⁴, 2500 and 250. The standard curve plot consists of the log of the starting copy number of the DNA standards versus the threshold cycle (Cₜ) value, which is a straight line. The Cₜ values are inversely proportional to the copy numbers values therefore the higher the copy number, the lower the Cₜ values.

QPCR reaction mixtures, the final concentration of forward and reverse Vaccinia primers were 900 nM each. The final optimized concentration of the Vaccinia TaqMan MGB probe as well as the internal control TaqMan MGB probe was determined to be 225 nM each. Primers and probes were synthesized by Applied Biosystems. TaqMan Universal 2×Master Mix (Applied Biosystems) was used in all QPCR reactions. Twelve microlitres of the 30 μL total eluant was analyzed per well. All reaction volumes used per well were 50 μL. The universal cycling parameters used were according to Applied Biosystems recommendations.

The Sequence Detection Software plots the standard curve and determines the slope, R value and the Y-intercept for the standard curve. A slope of −3.3 is indicative of 100% PCR efficiency whereby there is the expected 3.3 Cₜ difference based on a ten-fold dilution. The Y-intercept provides the PCR cycle that corresponds to the cycle where a single copy of the target can be detected. The PCR efficiency can be calculated using the formula: 10^{-1/S}−1 where S is the slope of the standard curve. The copy number in the unknowns is calculated by interpolation from the standard curve using the slope and the Y-intercept values.

Experiments with known numbers of live vaccinia virus strain Wyeth, added to 500 μL of vaccinia-negative plasma and DNA extracted by QIAamp® UltraSens™ Virus Kit (Qiagen) according to manufacturers instructions, proved the efficacy of the method in detecting vaccinia virus.

Ten VIG plasma mini-pools, a Cangene VIG bulk drug product (lot # 196.01.001), a Cangene VIG final product (lot # 0730203) were tested by the QPCR method. The standard curve consisted of the following points, 10⁶, 10⁵, 10⁴, 2500 and 250 copies resulting in Cₜ values of 17.54, 20.69, 23.73, 25.71 and 26.80 respectively. The standard curve had an R value of 0.944. The Cₜ values for the samples tested were greater than 30. Such results indicate all ten VIG plasma mini-pools, Cangene VIG bulk drug product and Cangene VIG final product to have less than 1250 copies of vaccinia virus genomes per milliliter.

Clinical Studies

Safety Study

The safety and pharmacokinetic characteristics of VIG-CAN-1 given in two intravenous doses will be determined. The VIG-CAN-1 is drawn up with an equivalent amount of Saline USP for injection such as the total volume of the solution is to equal to 0.6 mL/kg:
0.3 ml/kg of VIG-CAN-1 intravenously (n=25) with 0.3 ml/kg Saline USP or
0.6 ml/kg of VIG-CAN-1 intravenously (n=25)

The VIG-CAN-1 will be given intravenously via appropriate canula. After administration of the VIG-CAN-1, blood samples will be collected and adverse events recorded (at 30 min., 1, 2, 4, 8 and 24 hours). Subjects return over the course of six weeks on days 1, 3, 5, 7, 14, 21, 28, 42, to provide additional blood and urine samples for analysis, and for adverse event and concomitant medication reporting. A complete physical examination will be performed at screening and day 42.

Study Assessments

For the preparation of samples, 5 mL of blood is drawn into sterile, pyrogen-free, vacuum blood collection tubes using sodium heparin (15 U/ml blood) as anticoagulant.

Two 5 mL samples of plasma to be measured for vaccinia IgG levels by ELISA assay will be sent at the following times after study drug administration:
Time 0, (before administration of VIG-CAN-1)
30 min, 1 hr, 2 hr, 4 hr, 8 hr, 24 hr
Day 1 (same as 24 hr sample), 3, 5, 7, 14, 21, 28, 42

The following tests are conducted at Baseline, days 7, 28, and 42:
Hematology: including RBC, Hgb, Het, WBC, Differnetial cell counts, Platelet count, RBC indices, Prothrombin time, Partial thromboplastin time, Reticulocyte, Fibrinogen, ESR
Blood Chemistry calcium, Urea nitrogen, Glucose [random], Total Bilirubin, AST, ALT, Alkaline Phosphatase, Lactate Dehydrogenase, Creatinine, Sodium, Potassium, Chloride, Albumin, Total proteins
Urinalysis (Specific Gravity, pH, Protein, Glucose, Ketones, Bilirubin, and blood)
A full physical examination will be conducted at the 28^{th} and 42^{nd} day visit
The study subjects are questioned during all assessment visits following drug administration about adverse events they may be experiencing and concomitant medications they may be taking
Beta hCG [for premenopausal female subjects]
   Assessments for Safety

An adverse event (AE) is any untoward medical occurrence in a patient or clinical investigation subject. An AE includes significant exacerbation of any baseline medical condition including, but not limited to, the disease under study. The event must occur in association with drug treatment but a causal relationship to treatment is not necessarily proven or implied.

Data collected to date indicates intravenous injection of the VIG-CAN-1 at 6,000 U/Kg is safe in humans, with no adverse events reported at the 6-week follow-up visit.

Preclinical Studies

Mouse Tail Infectivity Model:

VIG has been indicated for prevention or modification of aberrant infections induced by the vaccinia virus vaccine. The following protocols employ a mouse tail infectivity model that involves inoculation of vaccinia virus into the murine tail vein. This model of infectivity closely resembles the human vaccination process in that dermal lesions occur on the tail similar to the vaccination site lesion that occurs in humans, and both result in a self-limiting infection. Therefore, this is a good surrogate model for VIG-CAN-1 efficacy testing with this indication.

A tail infectivity model for vaccinia is established using female Balb/c mice (Weight range 19 to 23 g) challenged with Vaccinia strain IHD-J administered by tail vein injection. These animals are challenged with 1×10⁵ PFU of virus in 200 μl total volume and the pox appearing on the tail scored for 7 days. The dose of virus leading to 50 pox per tail are determined and this dose will be used for subsequent challenge studies with vaccinia immune globulin (VIG-CAN-1).

The post exposure protection of VIG-CAN-1 is determined in the above described tail infectivity model. VIG-CAN-1 is tested in Balb/c mice challenged with a dose for vaccinia virus determined above to result in 50 pox per tail. Five doses of test VIG-CAN-1 is administered 24 hours after infection. After 7 days, the number of tail pocks is assessed by tail staining. The primary endpoint is the number of pocks per tail at Day 7. A dose response analysis is applied to the negative control, the five test VIG-CAN-1 groups and the five control VIG-CAN-1 groups.

Positive control is Cidofovir™ administered intramuscularly at 100 mg/Kg in a total volume of 100 μl. Negative control is Saline administered at 100 μl intramuscularly. VIG-CAN-1 administered at 6000 U/Kg and 30000 U/KG intramuscularly. VIG-CAN-1 (30000 U/Kg) and Cidofovir™ (100 mg/Kg) administered intramuscularly in a total volume of 100 μl.

Treatment with 6000 U/Kg of VIG resulted in similar number of pocks compared to the positive control. Vig 30000 and Vig 30000+ Cidofovir™ gave significantly fewer pocks compared to the positive control, as shown in Table 2.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1

| | Number of | | | | |
|---|---|---|---|---|---|
| | subjects | Incidence | Mean | | Peak |
| Adverse Events | (n = 20) | (n = 20) | Duration | | Occurrence |
| Redness | 20 | 100% | 14 | days | Day 7 |
| Swelling | 19 | 95% | 10 | days | Day 7 |
| Crusting | 19 | 95% | 7 | days | Day 14 |
| Itching | 18 | 90% | 8.5 | days | Day 7 |
| Pustule | 16 | 80% | 5 | days | Day 9 |
| Blistering | 13 | 65% | 5.5 | days | Day 2 |
| Pain | 10 | 50% | 3 | days | Day 7 |
| Headache | 8 | 40% | 1 | day | Day 2 |
| URI | 6 | 30% | 3 | days | Day 2 |
| Adhesive skin | 5 | 25% | 2 | days | Day 8 |
| reaction | | | | | |
| Fatigue | 4 | 20% | 7 | days | Day 7-9 |
| Other | 4 | 20% | 7 | days | Day 10 |
| Discharge at | 3 | 15% | 9 | days | Day 9 |
| vaccination site | | | | | |
| Myalgia | 3 | 15% | 3 | days | Day 11 |
| Nausea | 2 | 10% | 2 | days | Day 7 or 9 |
| Lymphadenopathy | 2 | 10% | 5 | days | Day 3 |
| Fever | 1 | 5% | 1 | day | Day 2 |
| Local Spread | 0 | 0% | | | |
| Distant Lesions | 0 | 0% | | | |
| Necrosis | 0 | 0% | | | |
| Vomiting | 0 | 0% | | | |

TABLE 2

| | | | | | Compared to positive control | | |
|---|---|---|---|---|---|---|---|
| | | | Compared to negative control | | | | p value for |
| | | | Mean | 95% Cl for | Mean | 95% Cl for | difference with |
| Treatment | Mean | 95% Cl | difference | difference | difference | difference | positive control |
| Saline | 41.9 | (36.7, 47.1) | | | | | |
| Cidofovir ™ | 24.2 | (21.0, 27.4) | −17.7 | (−23.3, −12.1) | | | |
| VIG 3000 | 23.7 | (21.4, 26.0) | −18.2 | (−23.5, −12.9) | −0.5 | (−4.2, 3.2) | p = 0.78 |
| VIG 6000 | 26.5 | (20.0, 33.0) | −15.4 | (−23.1, −7.7) | 2.3 | (−4.4, 9.0) | p = 0.48 |
| VIG 30000 | 16.9 | (14.3-19.5) | −27.3 | (−31.9, −22.7) | −7.7 | (−11.5, −3.9) | p < 0.001 |
| VIG 30000 + | 13.7 | (11.0-16.4) | −30.5 | (−35.1, −25.9) | −10.9 | (−14.7, −7.1) | p < 0.001 |
| Cidofovir ™ | | | | | | | |

What is claimed is:

1. A method of preparing an intravenously injectable immune globulin effective against Orthopoxvirus comprising:
vaccinating a plurality of donors with an Orthopoxvirus vaccine;
isolating plasma from each of said donors after a period of time sufficient to allow production of antibodies against said Orthopoxvirus vaccine;
pooling the plasma; and
preparing an intravenously injectable immune globulin from the pooled plasma.

2. The method according to claim 1 wherein prior to pooling the plasma, a titre of Orthopoxvirus antibodies in the plasma from each donor is determined and only plasma having titres above a threshold level are pooled.

3. The method according to claim 2 wherein the threshold level is 200 U/ml.

4. The method according to claim 1 wherein the intravenously injectable immune globulin can be safely administered to animals.

5. The method according to claim 1 wherein the intravenously injectable immune globulin is substantially free of blood borne pathogens.

6. The method according to claim 1 wherein the immune globulin is prepared from the pooled plasma by column isolation.

7. The method according to claim 6 wherein the column isolation is by ion exchange chromatography.

8. The method according to claim 2 wherein the titre of the plasma is determined by an ELISA assay.

9. The method according to claim 2 wherein the titre of the plasma is determined by a colorimetric assay.

10. The method according to claim 9 wherein the colorimetric assay comprises:
removing a sample of the plasma;
incubating the sample with a quantity of vaccinia virus;
adding the sample and vaccinia virus to cultured animal cells susceptible to vaccinia virus infection; and
adding a vital dye to the cultured animal cells.

11. A method of treating or preventing an Orthopoxvirus infection in an individual comprising administering intravenously to an individual in need of such treatment an intravenously injectable immune globulin effective against Orthopoxvirus.

12. The method according to claim 11 including administering an antiviral compound.

13. The method according to claim 12 wherein the antiviral compound is Cidofovir™.

14. The method according to claim 11 including administering an Orthopoxvirus vaccine.

15. A method of treating or ameliorating symptoms associated with adverse reaction to Orthopoxvirus vaccination comprising administering intravenously to an individual in need of such treatment an intravenously injectable immune globulin effective against Orthopoxvirus.

16. An intravenously injectable pharmaceutical composition comprising immune globulin effective against Orthopoxvirus.

17. The pharmaceutical composition according to claim 16 being substantially free of blood-borne pathogens.

18. The pharmaceutical composition according to claim 16 including a surfactant.

19. The pharmaceutical composition according to claim 16 including a stabilizer.

20. The pharmaceutical composition according to claim 16 including an Orthopoxvirus vaccine.

21. The pharmaceutical composition according to claim 16 including an antiviral pharmaceutical.

22. The pharmaceutical composition according to claim 21 wherein the antiviral pharmaceutical is Cidofovir™.

23. A colorimetric assay for measuring vaccinia virus neutralizing antibodies comprising:
adding a quantity of vaccinia virus to a sample suspected of containing vaccinia neutralizing antibodies, thereby forming a mixture;
incubating the mixture;
adding the mixture to cultured animal cells susceptible to vaccinia virus infection; and
adding a vital dye to the cells,
wherein the amount of neutralizing antibodies present in the sample is proportional to staining of the cultured cells by the vital dye.

24. A pharmaceutical kit for treatment of a Orthopoxvirus infection in a subject in need thereof, the kit comprising in packaged combination: an immune globulin effective against an Orthopoxvirus, and an antiviral agent effective against an Orthopoxvirus.

25. The kit according to claim 24 including instruction for use in treating an Orthopoxvirus Infection.

26. A method of testing a sample for vaccinia DNA comprising:
providing a sample suspected of containing vaccinia DNA under conditions suitable for oligonucleotide;
adding to said sample reagents needed for nucleotide amplification, said reagents including an enzyme capable of synthesizing nucleotide molecules, buffers and appropriate substrates for nucleotide synthesis, said reagents further comprising:
a forward primer comprising an oligonucleotide molecule binding to a first region of Vaccinia virus DNA;
a reverse primer comprising an oligonucleotide molecule binding to a second region of Vaccinia virus DNA, said second region being sufficiently proximal to the first region that an amplification product may be produced; and
a reporter primer comprising a reporter oligonucleotide molecule binding to a third region of Vaccinia virus DNA, said third region being between the first region and the second region, said reporter primer further comprising a reporter molecule attached to a first end of the reporter oligonucleotide and an effector molecule attached to a second end of the reporter oligonucleotide, said effector molecule altering a signal generated by the reporter molecule when the effector molecule and the reporter molecule are both attached to the reporter oligonucleotide; and
incubating the sample and reagents under conditions suitable for nucleotide amplification by the enzyme.

27. The method according to claim 26 wherein the forward primer is an oligonucleotide molecule comprising the nucleotide sequence of SEQ ID No. 1.

28. The method according to claim 26 wherein the reverse primer is an oligonucleotide molecule comprising the nucleotide sequence of SEQ ID No. 2.

29. The method according to claim 26 wherein the reporter oligonucleotide is an oligonucleotide comprising the nucleotide sequence of SEQ ID No. 3.
